(19) United States
(12) Patent Application Publication
(10) Pub. No.: US 2005/0125851 A1
(43) Pub. Date: Jun. 9, 2005

(54) Methods of diagnosis and treatment of interstitial lung disease

(75) Inventors: Jeffrey A. Whitsett, Cincinnati, OH (US); Stephan W. Glasser, Cincinnati, OH (US)

Correspondence Address: FROST BROWN TODD, LLC, 2200 PNC CENTER, 201 E. FIFTH STREET, CINCINNATI, OH, 45202, US

(73) Assignee: Children's Hospital Research Foundation, Cincinnati, OH (US)

(21) Appl. No.: 10/731,465

(22) Filed: Dec. 9, 2003

(51) Int. Cl. A01K 67/027

(52) U.S. Cl. 800/18

(57) ABSTRACT

The present invention provides for a method of treating pulmonary disease in a subject comprising the administration to a subject in need of such treatment a therapeutically effective amount of a formulation comprising a SP-C therapeutic. Preferably, the SP-C therapeutic is an agent selected from the group consisting of an isolated SP-C protein, an isolated nucleic acid molecule encoding a SP-C protein, a SP-C receptor-specific antibody that stimulates the activity of the receptor, or pharmaceutically acceptable composition thereof. The present invention also provides methods of producing a mouse with a targeted disruption in a surfactant protein C (SP-C) gene. The present invention also provides for a transgenic mouse produced by a targeted disruption in a surfactant protein C (SP-C) gene. The present invention further provides for a cell or cell line from a transgenic mouse produced by a targeted disruption in a surfactant protein C (SP-C) gene.

This application claims the benefit of U.S. Provisional Patent Application Ser. No. 60/431,949, filed Dec. 9, 2002, which application is hereby incorporated by reference in its entirety.

This invention was made with government support under Grant Nos. HL56387 and HL50046 awarded by the National Institutes of Health. The government has certain rights in the invention.

### BACKGROUND OF THE INVENTION

The present invention provides a mammal in which the expression of one or more lung surfactant protein genes has been suppressed. More particularly, the invention concerns the inactivating deletion of the surfactant protein C gene to produce a knockout non-human mammal with decreased or completely suppressed expression of the endogenous gene. The invention provides methods for preparing such knockout mammals and methods of using the knockout mammals to evaluate the effectiveness of therapeutic agents and regimens to treat diseases or disorders associated with perturbations in the lung surfactant protein pathways.

SP-C is a 34-35 amino acid peptide expressed selectively in type II epithelial cells in the alveolus of the lung [1,2 for review]. A single SP-C gene is located on human chromosome 8 that is syntenic to that in the mouse located on chromosome 14. The SP-C gene encodes a proprotein of 197 or 191 amino acids (proSP-C) that is palmitoylated, proteolytically processed and routed through the rough endoplasmic reticulum and multivesicular bodies to lamellar bodies in which surfactant is stored. The SP-C peptide is secreted into the airspace where it enhances the stability and spreading of phospholipids. The SP-C peptide is highly hydrophobic and also contains two cysteine residues in an NH₂-terminal domain. These cysteines are palmitoylated and located near an extended hydrophobic domain wherein 19 of 23 residues are valine, leucine or isoleucine. This hydrophobic region forms an α-helical structure that spans a lipid bilayer [3]. Both the α-helical domain and the cysteine linked palmitoyl groups are tightly associated with phospholipids. SP-C disrupts phospholipid acyl chain packing and enhances recruitment of phospholipids to monolayers and multilayers at the air-liquid interface [4,5]. These features suggest a structural role for SP-C in facilitating the movement of phospholipids between multilayered films. Biological functions of purified SP-C or synthetic SP-C peptides are highly active in vitro and in vivo, enhancing surfactant properties of lipids and restoring lung function in surfactant deficient animals [6,7]. These results indicate that SP-C plays an important role in the spreading and stabilization of phospholipid films in the alveolus.

An unexpected role for SP-C in pulmonary homeostasis was provided by recent studies demonstrating that a mutation in the SP-C gene was associated with idiopathic interstitial pneumonitis (IIP) in humans [8,9]. Pulmonary disease in these patients was inherited as an autosomal dominant trait. Interstitial pneumonitis includes various pulmonary disorders including desquamating interstitial pneumonitis (DIP), usual interstitial pneumonitis (UIP), nonspecific interstitial pneumonitis (NSIP), and other disorders broadly termed idiopathic interstitial pneumonitis (IIP) [10]. Individuals with these disorders usually present with progressive lung disease associated with exercise limitation, tachypnea and shortness of breath. Since mutations in the SP-C proprotein resulted in the production of an abnormal proSP-C peptide that was not fully processed, it has been unclear whether the lack of SP-C per se or misfolding of proSP-C or SP-C was involved in the pathogenesis of IIP in these patients [5]. In general, various forms of IIP are associated with alveolar inflammation, pulmonary infiltration with monocytesmacrophages, progressive loss of alveolar structure and pulmonary fibrosis [10]. The molecular mechanisms involved in the pathogenesis of IIP have been elusive in spite of well-recognized histologic and clinical manifestations.

There are two basic types of animals with genetically manipulated genomes. A traditional transgenic mammal has a modified gene introduced into its genome and the modified gene can be of exogenous or endogenous origin. A “knockout” mammal is a special type of transgenic mammal, characterized by suppression of the expression of an endogenous gene through genetic manipulation. The disruption of specific endogenous genes can be accomplished by deleting some portion of the gene or replacing it with other sequences to generate a null allele. Cross-breeding mammals having the null allele generates a homozygous mammals lacking an active copy of the gene.

A number of such mammals have been developed, and are extremely helpful in medical development. For example, U.S. Pat. No. 6,245,963, details a knockout-transgenic mouse model of spinal muscular atrophy and U.S. Pat. No. 6,414,219 details an osteopontin knockout mouse.

Transgenic animal models of SP-C mediated pulmonary diseases would be very useful for identifying pharmaceutical agents that are able to treat or prevent pulmonary diseases.

### BRIEF DESCRIPTION OF THE DRAWINGS

FIG. 1. Progression of pulmonary histopathology in SP-C (−/−) mice. Lungs were obtained from wild type littermates (A,C,E) or SP-C (−/−) (B,D,F) mice. Lungs were inflation fixed at 20 cm H₂O of pressure and stained with hematoxylin-eosin. Airspace enlargement, variable stromal thickening, and monocytic infiltration were noted at 2 months (B), 6 months (D), and 12 months (F) of age. Perivascular and peribronchiolar mononuclear infiltrates and epithelial cell dysplasia in conducting airways are shown in panel F. Micrographs 625× are representative of at least 3-5 animals of similar ages.

FIG. 2. Emphysema in SP-C (−/−) mice. Lung histology in control (panel A) and SP-C (−/−) mice (panel B) demonstrate the marked increase in alveolar size at one year of age. Macrophage infiltrates are observed in the central and lower regions of panel B.

FIG. 3. Remodeling and increased trichrome staining in lungs from SP-C (−/−) mice. Mason trichrome (A,B), orcein (C,D), and α-smooth muscle actin immunostaining (E,F) are shown from lung tissue at 6 months of age in wild type (A,C,E) and SP-C (−/−) (B,D,F) littermates. Airspace remodeling with monocytic infiltration and dense blue staining was observed (arrows). Orcein staining demonstrated that elastin fibers were absent in many of the remodeled airspaces (D). α-Smooth muscle actin staining was observed in alveolar regions of the lung parenchyma in SP-C (−/−) (arrows), but not wild type, mice.

FIG. 4. Ultrastructural abnormalities in lungs of SP-C (−/−) mice. Electronmicroscopy was performed on SP-C (−/−) mice at 9 months of age. Marked abnormalities were observed in the alveolar walls from the SP-C (−/−) mice (A). Type II cells were hyperplastic, containing numerous lamellar body-like inclusions, collagen deposition was noted within alveolar walls. Alveolar capillaries were surrounded by thickened subepithelial stroma. Conducting airways were lined by dysplastic epithelial cells with atypical morphology (B). Numerous cytopathic dense organelles, likely representing atypical mitochondria were observed in nonciliated columnar epithelial cells. Alveolar macrophages were hyperplastic, some containing dense crystals (top cell, C). Others containing excessive amounts of surfactant lipids, including lamellar bodies and tubular myelin figures, were observed. Pulmonary vascular abnormalities were observed in small vessels in SP-C (−/−) mice. Vessels were occluded or absent in many alveoli. Abnormal membrane blebbing was recurrently observed along the intima of the abnormal vessels (D).

FIG. 5. Alveolar macrophage infiltrates in the SP-C (−/−) mice. MAC-3 immunostaining was assessed in wild type (A) and SP-C (−/−) (B) mice at 6 months of age. Extensive infiltration with MAC-3 staining cells was noted in association with severe emphysema (B). Micrograph (625×) is representative of at least 5 SP-C (−/−) mice and controls. Semi-thin sections of wild type (C) and SP-C (−/−) (D) mice were stained with toluidine-blue, demonstrating alveolar and alveolar macrophage abnormalities. Lipid inclusions were noted in hyperplastic type II cells lining the alveoli and in the numerous alveolar macrophages accumulating in the airspaces.

FIG. 6. Epithelial cell dysplasia and MUC5A/C staining in conducting airways of 2 month old SP-C (−/−) mice. Conducting airways from wild type (A,C) or SP-C (−/−) (B,D) are observed after H&E staining (A,B) or MUC5A/C immunohistochemistry (C,D). Epithelial cell dysplasia was observed in large and small conducting airways of SP-C (−/−) mice. The abnormal epithelial cells were hypertrophic with abnormal foci of pseudostratified epithelia. While MUC5A/C staining cells were rarely seen in wild type mice (C), extensive staining for MUC5A/C was observed throughout bronchi and bronchioles (D), and was occasionally observed in the peripheral lung parenchyma in SP-C (−/−) mice (not shown). Panels A,B: 625× magnification; Panels C,D: 1250× magnification.

FIG. 7. Pressure-volume analysis demonstrates increased lung volumes in SP-C (−/−) mice. Pressure-volume curves were performed in tracheotomized wild type and SP-C (−/−) mice at 10-12 months of age, n=5 per group. Significantly increased lung volumes at higher pressure were observed in SP-C (−/−) mice, *p<0.01.

FIG. 8. Phospholipid (SatPC) and surfactant proteins in SP-C (−/−) mice. A: SatPC pool sizes were determined in wild type and SP-C (−/−) mice in BALF, lung tissue after BAL and the sum of BALF and tissue fractions (total). SatPC were increased 60% in BALF and 2-fold in tissue and total in SP-C (−/−) mice as compared to wild type mice at 15 months of age. B: Amounts of surfactant proteins in BALF were estimated by Western blot relative to the amount of SatPC. Values for wild type mice were normalized to a value of 1. SP-A and SP-D were increased in SP-C (−/−) mice. C: Pool sizes/body weight for SP-A, SP-B, and SP-D in BALF were normalized to a value of 1 for wild type mice. While SP-B levels were unaltered, SP-A and SP-D were increased in SP-C (−/−) mice. Mean±SE. *p<0.05.

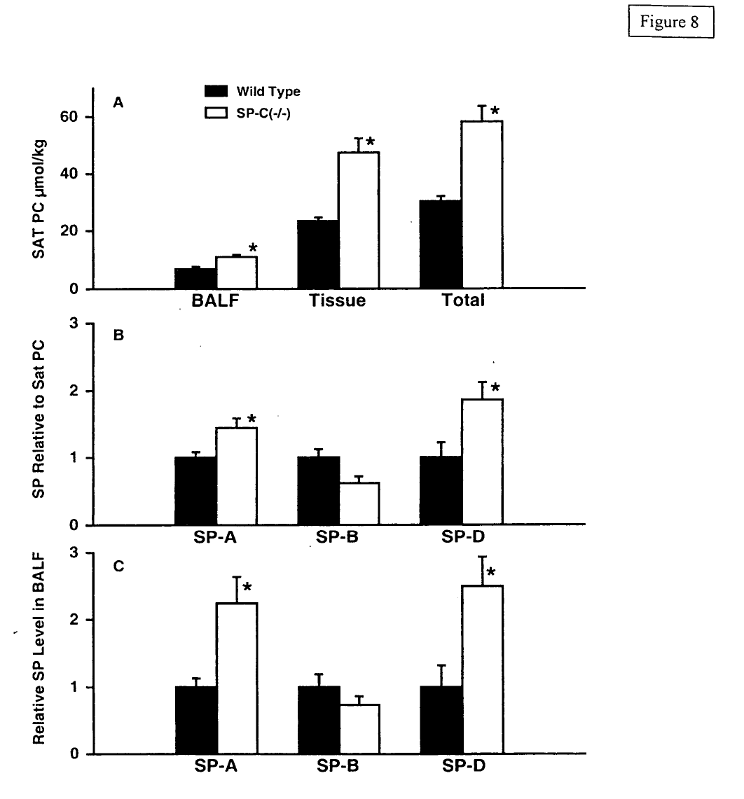

FIG. 9. Increased metalloproteinase activity produced by macrophages from SP-C (−/−) mice. MMP activity was assessed by zymography of conditioned media from alveolar macrophages from SP-C (−/−), lane 1 and SP-C (+/+), lane 2. Protease activity 72 kd (MMP-2) and 105 kd (MMP-9) were increased in media from SP-C (−/−) mice (arrows). A faint band at 55 kd, consistent with the size of MMP-12, was also increased in conditional media from SP-C (−/−) mice (arrowhead). Gels are consistent with observations from 4 separate experiments.

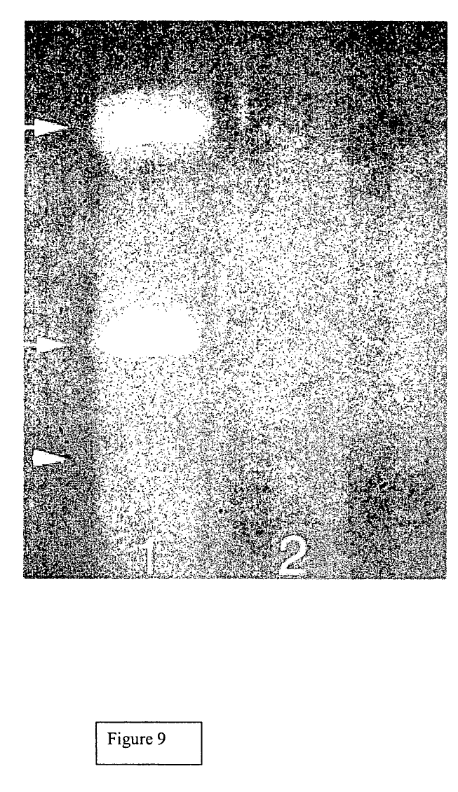

### STATEMENT OF THE INVENTION

The generation of SP-C (−/−) mice in a congenic 129JSV strain resulted in the surprising finding that genetic ablation of SP-C caused a progressive severe pulmonary fibrosis, expression of the mucin gene MUC5 in the conducting airways, epithelial cell dysplasia in conducting airways, emphysema, alveolar vascular remodeling, and right heart hypertrophy. Surprisingly, severe lung pathology developed in the absence of associated abnormalities in surfactant concentrations, and minimal alterations in surface properties of pulmonary surfactant isolated from the lung of SP-C (−/−) mice were observed. These findings demonstrate that a specific lack of SP-C/proSP-C per se, causes severe lung disease.

In humans bearing dominantly inherited gene mutations in SP-C (that causes the production of a misfolded proprotein, as well as disrupting the expression of the normal protein), a deficiency of proSP-C or SP-C per se also causes pulmonary disease. The pathology of the lung disease includes idiopathic pulmonary fibrosis (IPF), desquamating interstitial pneumonitis (DIP), usual interstitial pneumonitis (UIP), non-specific interstitial pneumonitis (NSIP), and other forms of interstitial lung disease.

The present invention provides for the use of a diagnostic screening based on the absence of SP-C or proSP-C in tissues or lavage lung material using immunohistochemistry, ELISA, Western blots, Mass spectroscopy, and protein sequencing.

The present invention also provides for replacement of proSP-C or SP-C, whether by gene transfer vectors to express the normal allele or protein replacement with purified SP-C, proSP-C or recombinant SP-C or recombinant proSP-C or SP-C or proSP-C analogues, are beneficial for the treatment of these pulmonary disorders. SP-C may be administered by aerosol or inhalation of a pharmaceutically useful preparation containing surfactant-like phospholipids, including phosphatidylglycerol, phosphatidylcholine.

The present invention also provides for SP-C (−/−) mice providing a model for testing therapies for interstitial lung disease, and for determining molecular pathways, activated or suppressed, that contribute to or cause the severe pulmonary disease seen in SP-C (−/−) mice.

The present invention provides methods of producing a mouse with a targeted disruption in a surfactant protein C (SP-C) gene. The present invention also provides for a transgenic mouse produced by a targeted disruption in a surfactant protein C (SP-C) gene. The present invention further provides for a cell or cell line from a transgenic mouse produced by a targeted disruption in a surfactant protein C (SP-C) gene. The present invention further provides for a surfactant protein C knock-out construct, comprising a portion of an surfactant protein C (SP-C) gene, wherein an internal portion of said SP-C gene is replaced by a selectable marker and at least 50 consecutive nucleotides of SP-C gene coding sequence have been deleted. Preferably, the SP-C deficient (SP-C −/−) mice develop a severe progressive pulmonary disorder with histologic features consistent with interstitial pneumonitis.

SP-C deficient mice developed severe, progressive pulmonary disease associated with emphysema, diffuse alveolar fibrosis, monocytic infiltrates, and epithelial cell dysplasia in conducting and peripheral airways. Targeted deletion of proSP-C in mice causes a syndrome similar to interstitial pneumonitis in humans.

In one aspect, the invention provides transgenic non-human organisms and cell lines for use in the in vivo screening and evaluation of drugs or other therapeutic regimens useful in the treatment of pulmonary disorders. In one embodiment, the invention is a transgenic animal with a targeted disruption in a pulmonary surfactant gene. In particular, the gene is the SP-C gene. The animal may be chimeric, heterozygotic or homozygotic for the disrupted gene. Homozygotic knockout SP-C mammals have a strong tendency towards developing a pulmonary condition, such as emphysema, monocytic infiltrates, fibrosis, epithelial cell dysplasia, and atypical accumulations of intracellular lipids in type II epithelial cells and alveolar macrophages. The targeted disruption may be anywhere in the gene, subject only to the requirement that it inhibit production of functional SP-C protein.

The DNA sequence of the mouse surfactant protein C (SP-C) gene (GenBank Acc. No. M38314) is shown in SEQ ID NO:1. The exonic DNA sequence of the mouse SP-C gene (GenBank Acc. No. M38314) is shown in SEQ ID NO:2. The polypeptide sequence of the mouse surfactant protein C (SP-C) (GenBank Acc. No. AAA40010) is shown in SEQ ID NO:3. The DNA sequence of the human surfactant protein C (SP-C) gene (GenBank Acc. No. J03890) is shown in SEQ ID NO:4. The polypeptide sequence of the human surfactant protein C (SP-C) (GenBank Acc. No. AAC32022) is shown in SEQ ID NO:5. The DNA sequence of the human surfactant protein Cl (SP-C1) (GenBank Acc. No. AAC32023) is shown in SEQ ID NO:6.

In a preferred embodiment, the disruption occurs within exon 2 of the wild type gene. In a more preferred embodiment, the disruption includes at least a disruption of nucleotide 1667 at the ApaL1 site in exon 2 of the wild type gene. The transgenic animal may be of any species (except human), but is preferably a mammal. In a preferred embodiment, the non-human animal comprising a targeted disruption in the surfactant protein C gene, wherein said targeted disruption inhibits production of wild-type surfactant protein C so that the phenotype of a non-human mammal homozygous for the targeted disruption is characterized by a pulmonary disorder condition.

In another aspect, the invention features a cell or cell line, which contains a targeted disruption in the surfactant protein C gene. In a preferred embodiment, the cell or cell line is an undifferentiated cell, for example, a stem cell, embryonic stem cell, oocyte or embryonic cell.

Yet in a further aspect, the invention features a method of producing a non-human mammal with a targeted disruption in a surfactant protein gene. For example, an SP-C knockout construct can be created with a portion of the SP-C gene having an internal portion of said SP-C gene replaced by a marker. The knockout construct can then be transfected into a population of embryonic stem (ES) cells. Transfected cells can then be selected as expressing the marker. The transfected ES cells can then be introduced into an embryo of an ancestor of said mammal. The embryo can be allowed to develop to term to produce a chimeric mammal with the knockout construct in its germline. Breeding said chimeric mammal will produce a heterozygous mammal with a targeted disruption in the SP-C gene. Homozygotes can be generated by crossing heterozygotes.

In another aspect, the invention features SP-C knockout constructs, which can be used to generate the animals described above. In one embodiment, the SP-C construct can comprise a portion of the surfactant protein C (SP-C) gene, wherein an internal portion of the gene is replaced by a selectable marker. Preferably, the marker is neomycin resistance gene and the portion of the SP-C gene is at least 2.5 kb long or 7.0 or 9.5 kb long (including the replaced portion and any SP-C flanking sequences). The internal portion preferably covers at least a portion of an exon and most preferably it is at least nucleotide 1667 at the ApaL1 site in exon 2 of the wild type gene.

In still another aspect, the invention features methods for testing agents for effectiveness in treating and/or preventing a pulmonary condition. In one embodiment, the method can employ the transgenic animal or cell lines, as described above. For example, a test agent can be administered to the transgenic animal and the ability of the agent to ameliorate the pulmonary condition can be scored as having effectiveness against said pulmonary condition. Any pulmonary condition with a surfactant component can be tested using these mammals, but in particular, conditions characterized by a lack of SP-C protein are studied. The method may also be used to test agents that are effective in replacing the SP-C pulmonary proteins and their downstream components.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are specifically incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

In the description that follows, a number of terms used in recombinant DNA technology are extensively utilized. In order to provide a clear and consistent understanding of the specification and claims, including the scope to be given such terms, the following definitions are provided.

The term “agonist”, as used herein, is meant to refer to an agent that mimics or upregulates (e.g. potentiates or supplements) SP-C bioactivity. An SP-C agonist can be a wild-type SP-C protein or derivative thereof having at least one bioactivity of the wild-type SP-C. An SP-C therapeutic can also be a compound that upregulates expression of an SP-C gene or which increases at least one bioactivity of the SP-C protein. Agonists can be any class of molecule, preferably a small molecule, including a nucleic acid, protein, carbohydrate, lipid or combination thereof.

“Antagonist” as used herein is meant to refer to an agent that down-regulates (e.g. suppresses or inhibits) at least one SP-C bioactivity. An antagonist can be a compound that down-regulates expression of an SP-C locus gene or that reduces the amount of an SP-C protein present. The SP-C antagonist can also be an SP-C antisense nucleic acid or a ribozyme capable of interacting specifically with SP-C RNA. Yet other SP-C antagonists are molecules that bind to SP-C polypeptide and inhibit its action. Such molecules include peptides. Yet other SP-C antagonists include antibodies interacting specifically with an epitope of an SP-C molecule, such that binding interferes with the biological function of the SP-C locus polypeptide.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene can differ from each other in a single nucleotide, or several nucleotides, and can include substitutions, deletions, and insertions of nucleotides. Frequently occurring sequence variations include transition mutations (i.e. purine to purine substitutions and pyrimidine to pyrimidine substitutions, e.g. A to G or C to T), transversion mutations (i.e. purine to pyrimidine and pyrimidine to purine substitutions, e.g. A to T or C to G), and alteration in repetitive DNA sequences (e.g. expansions and contractions of trinucleotide repeat and other tandem repeat sequences). An allele of a gene can also be a form of a gene containing a mutation. The term “allelic variant of a polymorphic region of an SP-C gene” refers to a region of an SP-C locus gene having one or several nucleotide sequence differences found in that region of the gene in other individuals.

As used herein, “pulmonary disease” refers to disorders and conditions generally recognized by those skilled in the art as related to the constellation of pulmonary diseases characterized by emphysema, monocytic infiltrates, fibrosis, epithelial cell dysplasia, and atypical accumulations of intracellular lipids in type II epithelial cells and alveolar macrophages, regardless of the cause or etiology. These include, but are not limited to, emphysema and interstitial pneumonitis.

“Biological activity” or “bioactivity” or “activity” or “biological function”, which are used interchangeably, for the purposes herein means a function that is directly or indirectly performed by an SP-C polypeptide (whether in its native or denatured conformation), or by any subsequence thereof. SP-C bioactivity can be modulated by directly affecting an SP-C polypeptide. Alternatively, an SP-C bioactivity can be modulated by modulating the level of an SP-C polypeptide, such as by modulating expression of an SP-C gene.

As used herein the term “bioactive fragment of an SP-C polypeptide” refers to a fragment of a full-length SP-C polypeptide, wherein the fragment specifically mimics or antagonizes the activity of a wild-type SP-C polypeptide.

The term “aberrant activity”, as applied to an activity of a polypeptide such as SP-C, refers to an activity which differs from the activity of the wild-type or native polypeptide or which differs from the activity of the polypeptide in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in an activity. A cell can have an aberrant SP-C activity due to overexpression or underexpression of an SP-C locus gene encoding an SP-C locus polypeptide.

“Cells”, “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric polypeptide” or “fusion polypeptide” is a fusion of a first amino acid sequence encoding one of the subject SP-C locus polypeptides with a second amino acid sequence defining a domain (e.g. polypeptide portion) foreign to and not substantially homologous with any domain of an SP-C polypeptide. A chimeric polypeptide may present a foreign domain that is found (albeit in a different polypeptide) in an organism that also expresses the first polypeptide, or it may be an “interspecies”, “intergenic”, etc. fusion of polypeptide structures expressed by different kinds of organisms. In general, a fusion polypeptide can be represented by the general formula X—SP-C—Y, wherein SP-C represents a portion of the polypeptide that is derived from an SP-C polypeptide, and X and Y are independently absent or represent amino acid sequences that are not related to an SP-C sequence in an organism, including naturally occurring mutants.

The phrase “nucleotide sequence complementary to the nucleotide sequence set forth in SEQ ID NO. x” refers to the nucleotide sequence of the complementary strand of a nucleic acid strand having SEQ ID NO. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID NO. x refers to the complementary strand of the strand having SEQ ID NO. x or to any nucleic acid having the nucleotide sequence of the complementary strand of SEQ ID NO. x. When referring to a single stranded nucleic acid having the nucleotide sequence SEQ ID NO. x, the complement of this nucleic acid is a nucleic acid having a nucleotide sequence which is complementary to that of SEQ ID NO. x. The nucleotide sequences and complementary sequences thereof are always given in the 5′ to 3′ direction.

As is well known, genes may exist in single or multiple copies within the genome of an individual. Such duplicate genes may be identical or may have certain modifications, including nucleotide substitutions, additions or deletions, which all still code for polypeptides having substantially the same activity. The term “DNA sequence encoding an SP-C polypeptide” may thus refer to one or more genes within a particular individual. Moreover, certain differences in nucleotide sequences may exist between individual organisms, which are called alleles. Such allelic differences may or may not result in differences in amino acid sequence of the encoded polypeptide yet still encode a polypeptide with the same biological activity.

The phrases “disruption of the gene” and “targeted disruption” or any similar phrase refers to the site specific interruption of a native DNA sequence so as to prevent expression of that gene in the cell as compared to the wild-type copy of the gene. The interruption may be caused by deletions, insertions or modifications to the gene, or any combination thereof.

The term “haplotype” refers to a set of alleles that are inherited together as a group (are in linkage disequilibrium). As used herein, haplotype is defined to include those haplotypes that occur at statistically significant levels (p<0.05). As used herein, the phrase “an SP-C haplotype” refers to a haplotype in the SP-C locus.

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. A degree of homology or similarity of amino acid sequences is a function of the number of amino acids, i.e. structurally related, at positions shared by the amino acid sequences. An “unrelated” or “non-homologous” sequence shares less than 40% identity, though preferably less than 25% identity, with one of the SP-C locus sequences of the present invention.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

The term “SP-C related” as used herein is meant to include all mouse and human genes related to the human SP-C locus genes on human chromosome 8.

Where the term “SP-C” is used in reference to a gene product or polypeptide, it is meant to refer to all gene products encoded by the surfactant protein C locus on human chromosome 8 and their corresponding mouse homologs.

The term “SP-C therapeutic” refers to various forms of SP-C polypeptides, as well as peptidomimetics, nucleic acids, or small molecules, which can modulate at least one activity of an SP-C polypeptide by mimicking or potentiating (agonizing) or inhibiting (antagonizing) the effects of a naturally-occurring SP-C polypeptide. An SP-C therapeutic that mimics or potentiates the activity of a wild-type SP-C polypeptide is a “SP-C agonist”. Conversely, an SP-C therapeutic that inhibits the activity of a wild-type SP-C polypeptide is an “SP-C antagonist”.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject SP-C polypeptides preferably includes no more than 5 kilobases (kb) of nucleic acid sequence which naturally immediately flanks the SP-C gene in genomic DNA, more preferably no more than 10 kb of such naturally occurring flanking sequences, and most preferably less than 5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state. The term “isolated” is also used herein to refer to polypeptides that are isolated from other cellular proteins and is meant to encompass both purified and recombinant polypeptides.

The term “knockout” refers to partial or complete suppression of the expression of an endogenous gene. This is generally accomplished by deleting a portion of the gene or by replacing a portion with a second sequence, but may also be caused by other modifications to the gene such as the introduction of stop codons, the mutation of critical amino acids, the removal of an intron junction, etc.

The term “knockout construct” refers to a nucleic acid sequence that can be used to decrease or suppress expression of a protein encoded by endogenous DNA sequences in a cell. In a simple example, the knockout construct is comprised of a gene, such as the SP-C gene, with a deletion in a critical portion of the gene so that active protein cannot be expressed therefrom. Alternatively, a number of termination codons can be added to the native gene to cause early termination of the protein or an intron junction can be inactivated. In a typical knockout construct, some portion of the gene is replaced with a selectable marker (such as the neo gene) so that the gene can be represented as follows: SP-C 5′/neo/SP-C 3′, where SP-C5′ and SP-C 3′, refer to genomic or cDNA sequences which are, respectively, upstream and downstream relative to a portion of the SP-C gene and where neo refers to a neomycin resistance gene. In another knockout construct, a second selectable marker is added in a flanking position so that the gene can be represented as: SP-C/neo/SP-C/TK, where TK is a thymidine kinase gene which can be added to either the SP-C5′ or the SP-C3′ sequence of the preceding construct and which further can be selected against (i.e. is a negative selectable marker) in appropriate media. This two-marker construct allows the selection of homologous recombination events, which removes the flanking TK marker, from non-homologous recombination events that typically retain the TK sequences. The gene deletion and/or replacement can be from the exons, introns, especially intron junctions, and/or the regulatory regions such as promoters.

The term “knockout mammal” and the like, refers to a transgenic mammal wherein a given gene has been suppressed by recombination with a knockout construct. It is to be emphasized that the term is intended to include all progeny generations. Thus, the founder animal and all F1, F2, F3, and so on, progeny thereof are included.

The term “chimera,” “mosaic,” “chimeric mammal” and the like, refers to a transgenic mammal with a knockout construct in some of its genome-containing cells.

The term “heterozygote” “heterozygotic mammal” and the like, refers to a transgenic mammal with a knockout construct on one of a chromosome pair in all of its genome-containing cells.

The term “homozygote” “homozygotic mammal” and the like, refers to a transgenic mammal with a knockout construct on both members of a chromosome pair in all of its genome-containing cells.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage equilibrium”.

The term “marker” or “marker sequence” or similar phrase means any gene that produces a selectable genotype or preferably a selectable phenotype. It includes such examples as the neo gene, green fluorescent protein (GFP) gene, TK gene, β-galactosidase gene, etc. The marker sequence may be any sequence known to those skilled in the art that serves these purposes, although typically the marker sequence will be a sequence encoding a protein that confers a selectable trait, such as an antibiotic resistance gene, or an enzyme that can be detected and that is not typically found in the cell. The marker sequence may also include regulatory regions such as a promoter or enhancer that regulates the expression of that protein. However, it is also possible to transcribe the marker using endogenous regulatory sequences. In one embodiment of the present invention, the marker facilitates separation of transfected from untransfected cells by fluorescence activated cell sorting, for example by the use of a fluorescently labeled antibody or the expression of a fluorescent protein such as GFP. Other DNA sequences that facilitate expression of marker genes may also be incorporated into the DNA constructs of the present invention. These sequences include, but are not limited to transcription initiation and termination signals, translation signals, post-translational modification signals, intron splicing junctions, ribosome binding sites, and polyadenylation signals, to name a few. The marker sequence may also be used to append sequence to the target gene. For example, it may be used to add a stop codon to truncate SP-C translation.

The use of selectable markers is well known in the art and need not be detailed herein. The term “modulation” as used herein refers to both upregulation (i.e., activation or stimulation (e.g., by agonizing or potentiating)) and downregulation (i.e. inhibition or suppression (e.g., by antagonizing, decreasing or inhibiting)).

The term “mutated gene” refers to an allelic form of a gene, which is capable of altering the phenotype of a subject having the mutated gene relative to a subject that does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant SP-C genes is present and/or expressed or disrupted in some tissues but not others. The term “non-human mammal” refers to any members of the class Mammalia, except for humans.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The term “polymorphism” refers to the coexistence of more than one form of a gene or portion (e.g., allelic variant) thereof A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

The terms “protein”, “polypeptide” and “peptide” are used interchangeably herein when referring to a gene product.

The term “recombinant protein” refers to a polypeptide of the present invention which is produced by recombinant DNA techniques, wherein generally, DNA encoding an SP-C polypeptide is inserted into a suitable expression vector which is in turn used to transform a host cell to produce the heterologous protein. Moreover, the phrase “derived from”, with respect to a recombinant SP-C gene, is meant to include within the meaning of “recombinant protein” those proteins having an amino acid sequence of a native SP-C polypeptide, or an amino acid sequence similar thereto which is generated by mutations including substitutions and deletions (including truncation) of a naturally occurring form of the polypeptide.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identify compounds that modulate an SP-C bioactivity.

As used herein, the term “specifically hybridizes” or “specifically detects” refers to the ability of a nucleic acid molecule of the invention to hybridize to at least approximately 6, 12, 20, 30, 50, 100, 150, 200, 300, 350, 400 or 425 consecutive nucleotides of a vertebrate, preferably an SP-C gene.

“Transcriptional regulatory sequence” is a generic term used throughout the specification to refer to DNA sequences, such as initiation signals, enhancers, and promoters, which induce or control transcription of protein coding sequences with which they are operably-linked. In preferred embodiments, transcription of one of the SP-C genes is under the control of a promoter sequence (or other transcriptional regulatory sequence) that controls the expression of the recombinant gene in a cell-type in which expression is intended. It will also be understood that the recombinant gene can be under the control of transcriptional regulatory sequences which are the same or which are different from those sequences which control transcription of the naturally-occurring forms of SP-C polypeptide.

As used herein, the term “transfection” means the introduction of a nucleic acid, e.g., via an expression vector, into a recipient cell by nucleic acid-mediated gene transfer. Methods for transformation that are known in the art include any electrical, magnetic, physical, biological or chemical means. As used herein, “transfection” includes such specific techniques as electroporation, magnetoporation, Ca⁺⁺ treatment, injection, bombardment, retroviral infection and lipofection, among others. “Transformation”, as used herein, refers to a process in which a cell's genotype is changed as a result of the cellular uptake of exogenous DNA or RNA, and, for example, the transformed cell expresses a recombinant form of an SP-C polypeptide or, in the case of anti-sense expression from the transferred gene, the expression of a naturally-occurring form of the SP-C polypeptide is disrupted.

As used herein, the term “transgene” means a nucleic acid sequence (encoding, e.g., one of the SP-C polypeptides, or an antisense transcript thereto) that has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the SP-C polypeptides, e.g. either agonistic or antagonistic forms. However, transgenic animals in which the recombinant SP-C gene is silent are also contemplated, as for example, the FLP or CRE recombinase dependent constructs described below. Moreover, “transgenic animal” also includes those recombinant animals in which gene disruption of one or more SP-C genes is caused by human intervention, including both recombination and antisense techniques.

The term “treating” as used herein is intended to encompass curing as well as ameliorating at least one symptom of the condition or disease.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The present invention provides for a method of treating pulmonary disease in a subject comprising the administration to a subject in need of such treatment a therapeutically effective amount of a formulation comprising a SP-C therapeutic. Preferably, the SP-C therapeutic is an agent selected from the group consisting of an isolated SP-C protein, an isolated nucleic acid molecule encoding a SP-C protein, a SP-C receptor-specific antibody that stimulates the activity of the receptor, or pharmaceutically acceptable composition thereof.

The present invention provides for the use of a SP-C therapeutic agent wherein the agent is a SP-C receptor-specific antibody that stimulates the activity of the receptor or wherein the agent is an isolated SP-C protein or proSP-C protein.

In one embodiment, the SP-C therapeutic agent is an isolated nucleic acid molecule encoding a SP-C protein or proSP-C protein, wherein the nucleic acid molecule is operatively linked to a transcription control sequence. Preferably, the nucleic acid molecule is expressed in the subject's airway cells. More preferably, the nucleic acid encodes a SP-C polypeptide, fragment, homolog or variant with substantial homology, supplying SP-C function.

In one embodiment, the nucleic acid molecule becomes integrated to the chromosomal DNA making up the genome of the subject's airway cells. In another embodiment, the nucleic acid molecule is expressed by the subject's airway cells from an extrachromosomal location. Generally, the nucleic acid molecule comprises at least 50 nucleotides. Preferably, the nucleic acid molecule comprises at least 200 nucleotides. The airway cells are generally smooth muscle and epithelial cells.

In one embodiment, the isolated nucleic acid molecule is administered to the mammal complexed with a liposome delivery vehicle. Alternatively, the isolated nucleic acid molecule is administered to the mammal in a viral vector delivery vehicle. Preferably, the viral vector delivery vehicle is from adenovirus.

In one embodiment, the isolated nucleic acid molecule, when administered to the lungs of the mammal, is expressed in cells of the mammal. Preferably, the disease is a chronic obstructive pulmonary disease of the airways associated with eosinophilic inflammation.

In another embodiment, the disease is selected from the group consisting of airway obstruction, allergies, asthma, acute inflammatory lung disease, chronic inflammatory lung disease, chronic obstructive pulmonary dysplasia, emphysema, pulmonary emphysema, chronic obstructive emphysema, adult respiratory distress syndrome, bronchitis, chronic bronchitis, chronic asthmatic bronchitis, chronic obstructive bronchitis, and interstitial lung diseases.

Preferably, the SP-C therapeutic agent decreases lung inflammation in the mammal. The SP-C therapeutic agent is administered in an amount between about 0.1 micrograms/kilogram and about 100 milligram/kilogram body weight of a mammal. Preferably in an amount between about 0.1 micrograms/kilogram and about 10 milligram/kilogram body weight of a mammal. In one embodiment, the SP-C therapeutic agent is administered in a pharmaceutically acceptable excipient.

The SP-C therapeutic agent may be administered by at least one route selected from the group consisting of nasal and inhaled routes.

In another embodiment, the pulmonary disease is selected from the group consisting of asthma, allergic bronchopulmonary aspergillosis, hypersensitivity pneumonia, eosinphilic pneumonia, allergic bronchitis bronchiectasis, hypersensitivity pneumotitis, occupational asthma, reactive airway disease syndrome, hypereosinophilic syndrome, rhinitis, sinusitis, and parasitic lung disease.

The present invention also provides for a method for prescribing treatment for airway hyperresponsiveness and/or airflow limitation associated with a respiratory disease involving an inflammatory response in a mammal, comprising: a. administering to the lungs of a mammal a SP-C therapeutic agent selected from the group consisting of: a SP-C receptor-specific antibody that stimulates the activity of the receptor an isolated SP-C protein or proSP-C protein; and an isolated nucleic acid molecule encoding a SP-C protein or proSP-C protein, wherein the nucleic acid molecule is operatively linked to a transcription control sequence; b. measuring a change in lung function in response to a provoking agent in the mammal to determine if the SP-C therapeutic agent modulates airway hyperresponsiveness; and c. prescribing a pharmacological therapy comprising administration of SP-C therapeutic agent to the mammal effective to reduce inflammation based upon the changes in lung function.

The present invention also provides for a formulation for protecting a mammal from airway hyperresponsiveness, airflow limitation and/or airway fibrosis associated with a respiratory disease involving inflammation, comprising an anti-inflammatory agent effective for reducing eosinophilic inflammation and a SP-C therapeutic agent selected from the group consisting of: a SP-C receptor-specific antibody that stimulates the activity of the receptor; an isolated SP-C protein or proSP-C protein; and an isolated nucleic acid molecule encoding a SP-C protein or proSP-C protein, wherein the nucleic acid molecule is operatively linked to a transcription control sequence. Generally, the formulation comprises a pharmaceutically acceptable excipient.

Preferably, the formulation comprises a controlled release vehicle selected from the group consisting of biocompatible polymers, other polymeric matrices, capsules, microcapsules, microparticles, bolus preparations, osmotic pumps, diffusion devices, liposomes, lipospheres, viral vectors and transdermal delivery systems.

In one embodiment, the SP-C therapeutic agent is an isolated SP-C protein or proSP-C protein. In another, the SP-C therapeutic agent is an isolated nucleic acid molecule encoding a SP-C protein or proSP-C protein, wherein the nucleic acid molecule is operatively linked to a transcription control sequence. In one embodiment, the isolated nucleic acid molecule is complexed with a liposome delivery vehicle. In another embodiment, the isolated nucleic acid molecule is provided in a viral vector delivery vehicle. Preferably, the viral vector delivery vehicle is from adenovirus.

In another embodiment, the SP-C therapeutic agent is a SP-C receptor-specific antibody that stimulates the activity of the receptor. In another embodiment, the SP-C therapeutic agent is selected from the group consisting of: an isolated SP-C protein or proSP-C protein and an isolated nucleic acid molecule encoding a SP-C protein or proSP-C protein, wherein the nucleic acid molecule is operatively linked to a transcription control sequence.

In another embodiment, the formulation may contain an anti-inflammatory agent selected from the group consisting of anti-IgE, immunomodulating drugs, leukotriene synthesis inhibitors, leukotriene receptor antagonists, glucocorticosteroids, steroid chemical derivatives, anti-cyclooxygenase agents, beta-adrenergic agonists, methylxanthines, cromones, anti-CD4 reagents, anti-IL-5 reagents, surfactants, cytoxin, and heparin. Preferably, anti-inflammatory agent is selected from the group consisting of leukotriene synthesis inhibitors, leukotriene receptor antagonists, glucocorticosteroids, beta-adrenergic agonists, methylxanthines, and cromones.

In general, the invention provides transgenic animals in which one or more SP-C related genes have been modified by transgenic cloning procedures. These SP-C transgenic animals are useful as animal models for various diseases that involve SP-C mediated pulmonary processes. Although most of the above described pulmonary diseases and conditions appear to have a complex and multifactorial etiology, they all appear to ultimately involve SP-C mediated pulmonary processes. The present invention also provides reagents and methods for the discovery of pharmaceutical compounds that are able to interfere with these SP-C mediated pulmonary processes and thereby block the progression of these otherwise disparate diseases.

In a preferred embodiment, the invention features a transgenic “knockout” mouse line in which the mouse SP-C (surfactant protein C) gene carried on mouse chromosome 14 at position 8p,8 is disrupted or deleted so as to decrease or eliminate expression of the SP-C gene.

The SP-C knockout mouse line features an enhancement of SP-C- mediated pulmonary disorder processes due to loss of endogenous SP-C protein molecules. In a further embodiment, the invention provides a “double” knockout mouse line featuring decreased expression of both the SP-C gene and at least one gene of SP-A, SP-B, or SP-D.

The transgenic “knockout” mouse line is useful to generate both heterozygous and homozygous SP-C gene knockout mice which can be used to study SP-C mediated pulmonary diseases and conditions. For example, loss of the surfactant protein C protein leads to increased SP-C mediated pulmonary disorders, and this contributes to the etiology of a number of diseases and conditions including: emphysema, monocytic infiltrates, fibrosis, epithelial cell dysplasia, and atypical accumulations of intracellular lipids in type II epithelial cells and alveolar macrophages.

Both chronic and acute forms of such pulmonary diseases and conditions can be reproduced in appropriate SP-C knockout animals or animal lines. For example, animals heterozygous for the SP-C knockout construct have diminished capacity to produce the surfactant protein C and therefore show a corresponding accentuation of SP-C mediated pulmonary processes. The heterozygous mouse lines may therefore reproduce the circumstances of chronic pulmonary diseases and conditions. Furthermore, these heterozygous animals or cell lines are well suited to finding therapeutic agents which act to accentuate the expression or activity of the diminished pool of endogenous surfactant protein C. Such receptor antagonist “agonists” may, for example, increase expression of the remaining copy of the SP-C gene. In contrast, homozygous SP-C “knockout” animals and lines have no ability to produce the SP-C gene product and hence show a correspondingly large enhancement of SP-C mediated processes. The homozygous animals and cell lines may therefore reproduce the aberrant pulmonary functions that occur in acute pulmonary diseases. Furthermore, these homozygous animals and lines are especially well suited to finding therapeutic agents that function, for example, as molecular mimics of the surfactant protein C by, for example.

The invention further provides various nucleic acid constructs useful for creating SP-C “knockout” and SP-C “knock-in” transgenic mouse cell lines and transgenic mice.

For example, an SP-C disrupting construct can be engineered so as to incorporate a reporter or marker gene (such as beta-galactosidase or green fluorescent protein) into a chromosomal copy of the gene, thereby rendering the resulting chimeric reporter gene dependent upon the endogenous SP-C gene promoter for its expression. Transgenic cell lines and animals incorporating such “knock-in” constructs are particularly well suited to the screening of compounds for their ability to suppress SP-C dependent pulmonary processes by increasing the transcription of the surfactant protein C gene. In another example, a heterologous regulatable promoter can be “knocked-in” to the SP-C gene locus so that surfactant protein C expression is now controlled by the regulatable promoter. The regulatable promoter can be an inducible promoter, a repressible promoter or a developmentally regulated promoter. The choice of promoters in this instance can be tailored to the specific study at hand. For example, a repressible promoter system facilitates the production of mouse lines in which the SP-C gene is expressed until some point in time after normal growth and development. The function of the SP-C gene can then be abruptly halted by administration of an appropriate ligand (such as tetracycline) which results in the transcriptional shut-down of the SP-C gene. This inducible surfactant protein C deficiency thereby triggers SP-C mediated pulmonary conditions in an otherwise normally developing animal. Pharmaceutical screens can thus be devised for compounds capable of blocking an surfactant protein C deficiency-induced pulmonary response.

The SP-C transgenic animals and cell lines of the present invention may thus be used for the development of pharmaceutical agents which are useful for treating or preventing such SP-C mediated diseases and conditions.

The gene to be knocked out may be any gene involved in the SP-C pathway, provided that at least some sequence or mapping information on the DNA to be disrupted is available to use in the preparation of both the knockout construct and the screening probes. In a preferred embodiment of the invention, the mouse SP-C gene on chromosome 14 at position 8p,8 is targeted for disruption. The genomic DNA sequence of the murine SP-C gene is shown in SEQ ID NO:1. These target gene constructs include SP-C target gene knockout and knock-in constructs which are specifically adapted to each of the various embodiments of the invention.

Important aspects of the present invention concern the disruption of genes, that express one or more SP-C polypeptides, generally having the sequences of mouse (GenBank accession number M38314; SEQ ID NO:1) or human (GenBank accession number J03890; SEQ ID NO:4) SP-C genes, or functional equivalents thereof. “Genes” refers to a DNA segment including any of the SP-C gene coding sequences and, in certain aspects, regulatory sequences, isolated substantially away from other naturally occurring genes or protein encoding sequences. In this respect, the term “gene” is used for simplicity to refer to a functional protein, polypeptide or peptide encoding unit. As will be understood by those in the art, this functional term includes both genomic sequences, cDNA sequences and smaller engineered gene segments that express, or may be adapted to express, proteins, polypeptides, domains, peptides or fusion proteins.

In particular embodiments, the invention concerns DNA sequences that encode an SP-C polypeptide that includes within its amino acid sequence a contiguous amino acid sequence of the mouse (GenBank accession number AAA40010; SEQ ID NO:3) or human (GenBank accession numbers AAC32022; SEQ ID NO:5 and AAC32023; SEQ ID NO:6) SP-C and SP-C1 polypeptides, respectively, or functional equivalents thereof.

Naturally, where the DNA segment encodes an SP-C polypeptide, or is intended for use in expressing the SP-C polypeptide, the most preferred sequences are those that are essentially as set forth in the contiguous sequence of SEQ I) NO:1, SEQ ID NO:2 or SEQ ID NO:4, and that encode a protein that retains SP-C biological activity. Sequence of an SP-C polypeptide will substantially correspond to a contiguous portion of that shown in SEQ ID NO:3, SEQ ID NO:5 or SEQ ID NO:6, and have relatively few amino acids that are not identical to, or a biologically functional equivalent of, the amino acids shown in SEQ ID NO:3, SEQ ID NO:5 or SEQ ID NO:6. The term “biologically functional equivalent” is well understood in the art and is further defined in detail herein.

Accordingly, sequences that have between about 70% and about 80%; or more preferably, between about 81% and about 90%; or even more preferably, between about 91% and about 99%; of amino acids that are identical or functionally equivalent to the amino acids of SEQ ID NO:3, SEQ ID NO:5 or SEQ ID NO:6 will be sequences that are “essentially as set forth in SEQ ID NO:3, SEQ ID NO:5 or SEQ ID NO:6.

In certain other embodiments, the invention concerns isolated DNA segments and recombinant vectors that include within their sequence a contiguous nucleic acid sequence from that shown in SEQ ID NO:1 SEQ ID NO:2, or SEQ ID NO:4. This definition is used in the same sense as described above and means that the nucleic acid sequence substantially corresponds to a contiguous portion of that shown in SEQ ID NO:1 SEQ ID NO:2, or SEQ ID NO:4, and has relatively few codons that are not identical, or functionally equivalent, to the codons of SEQ ID NO:1 SEQ ID NO:2, or SEQ ID NO:4. The term “functionally equivalent codon” is used herein to refer to codons that encode the same amino acid and also refers to codons that encode biologically equivalent amino acids.

Excepting intronic or flanking regions, and allowing for the degeneracy of the genetic code, sequences that have between about 70% and about 79%; or more preferably, between about 80% and about 89%; or even more preferably, between about 90% and about 99% of nucleotides that are identical to the nucleotides shown in the sequences of SEQ ID NO:1 SEQ ID NO:2, or SEQ ID NO:4 will be sequences that are “essentially as set forth in SEQ ID NO:1 SEQ ID NO:2, or SEQ ID NO:4”. Sequences that are essentially the same as those set forth in SEQ ID NO:1 SEQ ID NO:2, or SEQ ID NO:43 may also be functionally defined as sequences that are capable of hybridizing to a nucleic acid segment containing the complement of SEQ ID NO:1 SEQ ID NO:2, or SEQ ID NO:4 under relatively stringent conditions. Suitable relatively stringent hybridization conditions will be well known to those of skill in the art.

Naturally, the present invention also encompasses DNA segments that are complementary, or essentially complementary, to the sequence set forth in SEQ ID NO:1 SEQ ID NO:2, or SEQ ID NO:4. Nucleic acid sequences that are “complementary” are those that are capable of base- pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:1 or SEQ ID NO:3 under relatively stringent conditions.

It will also be understood that this invention is not limited to the particular nucleic acid and amino acid sequences of SEQ ID NO:1, SEQ ID NO:2, SEQ ID NO:3, SEQ ID NO:4, SEQ ID NO:5, or SEQ ID NO:6. DNA segments may therefore variously include the SP-C coding regions themselves, coding regions bearing selected alterations or modifications in the basic coding region, or they may encode larger polypeptides that nevertheless include SP-C-coding regions or may encode biologically functional equivalent proteins or peptides that have variant amino acids sequences.

The DNA segments of the present invention encompass biologically functional equivalent SP-C proteins and peptides. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes may be introduced through the application of site-directed mutagenesis techniques, e.g., to introduce improvements to the antigenicity of the protein or to test SP-C mutants in order to examine adenosine deaminase activity at the molecular level.

As modifications and changes may be made in the structure of SP-C genes and proteins of the present invention, and still obtain molecules having like or otherwise desirable characteristics, such biologically functional equivalents are also encompassed within the present invention.

For example, certain amino acids may be substituted for other amino acids in a protein structure without appreciable loss of interactive binding capacity with structures such as, for example, antigen-binding regions of antibodies, binding sites on substrate molecules or receptors, DNA binding sites, or such like. Since it is the interactive capacity and nature of a protein that defines that protein's biological functional activity, certain amino acid sequence substitutions can be made in a protein sequence (or, of course, its underlying DNA coding sequence) and nevertheless obtain a protein with like (agonistic) properties. It is thus contemplated by the inventors that various changes may be made in the sequence of SP-C proteins or polypeptides, or underlying DNA, without appreciable loss of their biological utility or activity.

In terms of functional equivalents, it is well understood by the skilled artisan that, inherent in the definition of a “biologically functional equivalent protein or peptide or gene”, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted.

In particular, where shorter length peptides are concerned, it is contemplated that fewer amino acid substitutions should be made within the given peptide. Longer domains may have an intermediate number of changes. The full length protein will have the most tolerance for a larger number of changes. Of course, a plurality of distinct proteins/peptides with different substitutions may easily be made and used in accordance with the invention.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In one embodiment, the clone to be used in producing the knockout construct is digested with a restriction enzyme selected to cut at a location(s) such that a marker gene can be inserted at that location in the gene. In alternative embodiments, DNA sequences can be removed by partial digestion with a random nuclease at a single restriction enzyme cut in the gene.

The proper position for marker gene insertion is that which will serve to prevent expression of the native gene. This position will depend on various factors, including which sequences (exon, intron or promoter) are to be targeted (i.e., the precise location of insertion necessary to inhibit promoter function or to inhibit synthesis of the native exon) and the availability of convenient restriction sites within the sequence. In some cases, it is desirable to remove a large portion of the gene so as to keep the length of the knockout construct comparable to the original genomic sequence when a marker gene is to be inserted into the knockout construct.

The marker gene can be any nucleic acid sequence well known to those skilled in the art and that is detectable and/or assayable. Typically, an antibiotic resistance gene is used or any other gene whose expression or presence in the genome can easily be detected. The marker gene is usually operably linked to a promoter from any source that will be active or can easily be activated in the cell into which it is inserted. However, the marker gene need not have its own promoter as it may be transcribed using the promoter of the targeted gene. In addition, the marker gene will normally have a polyA sequence attached to the 3′ end of the gene for transcription termination of the gene. Preferred marker genes are aminoglycoside phosphotransferase gene (aph), the hygromycin B phosphotransferase gene, or any antibiotic resistance gene known to be useful as a marker in knockout techniques.

The linear knockout construct may be transfected directly into embryonic stem cells (discussed below), or it may first be placed into a suitable vector for amplification prior to insertion. Suitable vectors are known to those skilled in the art.

The invention further provides for transgenic animals, which can be used for a variety of purposes, e.g., to identify therapeutics agents for SP-C mediated pulmonary disorders. The transgenic animals can be useful, e.g., for identifying drugs that modulate production of SP-C, such as by modulating SP-C gene expression. An SP-C gene promoter can be isolated, e.g., by screening of a genomic library with an SP-C cDNA fragment and characterized according to methods known in the art. In a preferred embodiment of the present invention, the transgenic animal containing said SP-C reporter gene is used to screen a class of bioactive molecules known as steroid hormones for their ability to modulate SP-C expression. In a more preferred embodiment of the invention, non-human animals are produced where the expression of the endogenous SP-C gene has been mutated or “knocked out”. A “knock out” animal is one carrying a homozygous or heterozygous deletion of a particular gene or genes. These animals could be useful to determine whether the absence of SP-C will result in a specific phenotype, in particular whether these mice have or are likely to develop a specific disease, such as high susceptibility to emphysema. Furthermore these animals are useful in screens for drugs that alleviate or attenuate the disease condition resulting from the mutation of the SP-C gene as outlined below. In a preferred embodiment of this aspect of the invention, a transgenic SP-C knock-out mouse, carrying the mutated SP-C locus on both of its chromosomes, is used as a model system for transgenic or drug treatment of the condition resulting from loss of SP-C expression.

Methods for obtaining transgenic and knockout non-human animals are well known in the art. In a general aspect, a transgenic animal is produced by the integration of a given transgene into the genome in a manner that permits the expression of the transgene, or by disrupting the wild-type gene, leading to a knockout of the wild-type gene. U.S. Pat. No. 5,616,491, incorporated herein by reference in its entirety, generally describes the techniques involved in the preparation of knockout mice.

Methods for producing transgenic animals are generally described in U.S. Pat. Nos. 4,736,866; 4,873,191; 5,175,383; 5,824,837; 6,437,216; 6,437,215; 6,374,130, which are incorporated herein by reference in their entirety. U.S. Pat. Nos. 5,639,457, 5,175,384; 5,175,385; 5,530,179, 5,625,125, 5,612,486 and 5,565,186 are also each incorporated herein by reference to similarly supplement the present teaching regarding transgenic pig, rabbit, mouse and rat production.

Knock out mice are generated by homologous integration of a “knock out” construct into a mouse embryonic stem cell chromosome that encodes the gene to be knocked out. In one embodiment, gene targeting, which is a method of using homologous recombination to modify an animal's genome, can be used to introduce changes into cultured embryonic stem cells. By targeting a SP-C gene of interest in ES cells, these changes can be introduced into the germlines of animals to generate chimeras. The gene targeting procedure is accomplished by introducing into tissue culture cells a DNA targeting construct that includes a segment homologous to a target SP-C locus, and which also includes an intended sequence modification to the SP-C genomic sequence (e.g., insertion, deletion, point mutation). The treated cells are then screened for accurate targeting to identify and isolate those that have been properly targeted.

Gene targeting in embryonic stem cells is in fact a scheme contemplated by the present invention as a means for disrupting a SP-C gene function through the use of a targeting transgene construct designed to undergo homologous recombination with one or more SP-C genomic sequences. The targeting construct can be arranged so that, upon recombination with an element of a SP-C gene, a positive selection marker is inserted into (or replaces) coding sequences of the gene. The inserted sequence functionally disrupts the SP-C gene, while also providing a positive selection trait. Exemplary SP-C targeting constructs are described in more detail below.

Generally, the embryonic stem cells (ES cells) used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al. (1985) J. Embryol. Exp. Mol. Biol. 87:27-45). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is preferred for production of ES cells, is the 129/Sv strain. The cells are cultured and prepared for knockout construct insertion using methods well known to the skilled artisan.

A typical knockout construct contains nucleic acid fragments of not less than about 0.5 kb nor more than about 10.0 kb from both the 5′ and the 3′ ends of the genomic locus which encodes the gene to be mutated. These two fragments are separated by an intervening fragment of nucleic acid that encodes a positive selectable marker, such as the neomycin resistance gene (neoR). The resulting nucleic acid fragment, consisting of a nucleic acid from the extreme 5′ end of the genomic locus linked to a nucleic acid encoding a positive selectable marker which is in turn linked to a nucleic acid from the extreme 3′ end of the genomic locus of interest, omits most of the coding sequence for SP-C or other gene of interest to be knocked out. When the resulting construct recombines homologously with the chromosome at this locus, it results in the loss of the omitted coding sequence, otherwise known as the structural gene, from the genomic locus. A stem cell in which such a rare homologous recombination event has taken place can be selected for by virtue of the stable integration into the genome of the nucleic acid of the gene encoding the positive selectable marker and subsequent selection for cells expressing this marker gene in the presence of an appropriate drug (neomycin in this example).

Variations on this basic technique also exist and are well known in the art. For example, a “knock-in” construct refers to the same basic arrangement of a nucleic acid encoding a 5′ genomic locus fragment linked to nucleic acid encoding a positive selectable marker which in turn is linked to a nucleic acid encoding a 3′ genomic locus fragment, but which differs in that none of the coding sequence is omitted and thus the 5′ and the 3′ genomic fragments used were initially contiguous before being disrupted by the introduction of the nucleic acid encoding the positive selectable marker gene. This “knock-in” type of construct is thus very useful for the construction of mutant transgenic animals when only a limited region of the genomic locus of the gene to be mutated, such as a single exon, is available for cloning and genetic manipulation. Alternatively, the “knock-in” construct can be used to specifically eliminate a single functional domain of the targeted gene, resulting in a transgenic animal that expresses a polypeptide of the targeted gene that is defective in one function, while retaining the function of other domains of the encoded polypeptide. In a variation of the knock-in technique, a marker gene is integrated at the genomic locus of interest such that expression of the marker gene comes under the control of the transcriptional regulatory elements of the targeted gene. A marker gene is one that encodes an enzyme whose activity can be detected (e.g., β-galactosidase), the enzyme substrate can be added to the cells under suitable conditions, and the enzymatic activity can be analyzed. One skilled in the art will be familiar with other useful markers and the means for detecting their presence in a given cell. For example, one such alternative marker is the green fluorescent protein (GFP). The GFP marker is particularly useful for the examination of gene expression in individual viable cells. Thus GFP and related markers are particularly useful for in situ analysis of levels of expression of the “knocked-in” gene. All such markers are contemplated as being included within the scope of the teaching of this invention.

Non-homologous recombination events can be selected against by modifying the above-mentioned knock out and knock in constructs so that they are flanked by negative selectable markers at either end (particularly through the use of two allelic variants of the thymidine kinase gene, the polypeptide product of which can be selected against in expressing cell lines in an appropriate tissue culture medium well known in the art—i.e. one containing a drug such as 5-bromodeoxyuridine). Thus a preferred embodiment of such a knock out or knock in construct of the invention consist of a nucleic acid encoding a negative selectable marker linked to a nucleic acid encoding a 5′ end of a genomic locus linked to a nucleic acid of a positive selectable marker which in turn is linked to a nucleic acid encoding a 3′ end of the same genomic locus which in turn is linked to a second nucleic acid encoding a negative selectable marker Nonhomologous recombination between the resulting knock out construct and the genome will usually result in the stable integration of one or both of these negative selectable marker genes and hence cells which have undergone nonhomologous recombination can be selected against by growth in the appropriate selective media (e.g. media containing a drug such as 5-bromodeoxyuridine for example). Simultaneous selection for the positive selectable marker and against the negative selectable marker will result in a vast enrichment for clones in which the knock out construct has recombined homologously at the locus of the gene intended to be mutated. The presence of the predicted chromosomal alteration at the targeted gene locus in the resulting knock out stem cell line can be confirmed by means of Southern blot analytical techniques, which are well known to those familiar in the art. Alternatively, PCR can be used.

Each knockout construct to be inserted into the cell must first be in the linear form. Therefore, if the knockout construct has been inserted into a vector (described infra), linearization is accomplished by digesting the DNA with a suitable restriction endonuclease selected to cut only within the vector sequence and not within the knockout construct sequence.

For insertion, the knockout construct is added to the ES cells under appropriate conditions for the insertion method chosen, as is known to the skilled artisan. For example, if the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knock out construct as explained above. Where more than one construct is to be introduced into the ES cell, each knockout construct can be introduced simultaneously or one at a time.

After suitable ES cells containing the knockout construct in the proper location have been identified by the selection techniques outlined above, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the knockout construct into the developing embryo. For instance, the transformed ES cells can be microinjected into blastocytes.

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the SP-C gene knocked out in various tissues of the offspring by probing the Western blot with an antibody against the particular SP-C protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Recombinase dependent knockouts can also be generated, e.g. by homologous recombination to insert target sequences, such that tissue specific and/or temporal control of inactivation of a SP-C-gene can be controlled by recombinase sequences.

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment, either the cre/loxP recombinase system of bacteriophage P1 or the FLP recombinase system of Saccharomyces cerevisiae can be used to generate in vivo site-specific genetic recombination systems, as known in the art. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present; catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements that are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant SP-C protein can be regulated via control of recombinase expression.

Use of the cre/loxP recombinase system to regulate expression of a recombinant SP-C protein requires the construction of a transgenic animal containing transgenes encoding both the Cre recombinase and the subject protein. Animals containing both the Cre recombinase and a recombinant SP-C gene can be provided through the construction of “double” transgenic animals. A convenient method for providing such animals is to mate two transgenic animals each containing a transgene, e.g., a SP-C gene and recombinase gene.

In an exemplary embodiment, introducing transgenes into the germline of the non-human animal produces the “transgenic non-human animals” of the invention. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenic, and/or may be knockouts (i.e., obtained from animals that have one or more genes partially or completely suppressed).

In one embodiment, the transgene construct is introduced into a single stage embryo. The zygote is the best target for microinjection. In the mouse, the male pronucleus reaches the size of approximately 20 micrometers in diameter, which allows reproducible injection of 1-2pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention, a zygote is essentially the formation of a diploid cell that is capable of developing into a complete organism. Generally, the zygote will be comprised of an egg containing a nucleus formed, either naturally or artificially, by the fusion of two haploid nuclei from a gamete or gametes. Thus, the gamete nuclei must be ones that are naturally compatible, i.e., ones that result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material, which can be added to the nucleus of the zygote or to the genetic material, which forms a part of the zygote nucleus. If no genetic material is removed, then the amount of exogenous genetic material, which can be added, is limited by the amount that will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs that are added to the zygote is dependent upon the total amount of exogenous genetic material added and will be the amount that enables the genetic transformation to occur. Theoretically only one copy is required, however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence, which results in the production of a SP-C protein (either agonistic or antagonistic), and antisense transcript, or a SP-C mutant. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et a l. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells that formed the transgenic non-human animal. Further, the founder may contain various retroviral insertions of the transgene at different positions in the genome, which generally will segregate in the offspring. In addition, it is also possible to introduce transgenes into the germ line by intrauterine retroviral infection of the mid-gestation embryo (Jahner et al. (1982) supra).

A third type of target cell for transgene introduction is the embryonal stem cell (ES). ES cells are obtained from pre-implantation embryos cultured in vitro and fused with embryos. Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal.

Pharmaceutical Screens

The invention provides various transgenic cell lines and organisms in which pharmaceutical screens can be conducted to identify compounds capable of effecting SP-C mediated pulmonary processes. As set forth above, the transgenic cell lines and organisms are engineered to be deficient in endogenous SP-C gene activities. The resultant loss of endogenous SP-C activity generally leads to an “acute phase response.” The acute phase response initiates further pulmonary processes, including those distinctive of the various pulmonary diseases and conditions discussed above.

Compounds identified above as being useful for preventing SP-C mediated pulmonary processes, can be, e.g. a nucleic acid (e.g. DNA, RNA or PNA), protein, peptide, peptidomimetic, small molecule, or derivative thereof Preferred compounds are capable of binding to, and regulating transcription, translation, processing, or activity of an SP-C gene or protein. Examples include antisense, ribozyme or triplex nucleic acids, small molecule ligands, antibody or antibody-like binding fragments.

Toxicity and therapeutic efficacy of such compounds can be determined by standard pharmaceutical procedures in cell cultures or experimental animals, e.g., for determining the LD₅₀ (The Dose Lethal To 50% Of The Population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Other suitable delivery systems include microspheres which offer the possibility of local noninvasive delivery of drugs over an extended period of time, This technology utilizes microspheres of precapillary size which can be injected via a coronary catheter into any selected part of the e.g. heart or other organs without causing inflammation or ischemia. The administered therapeutic is slowly released from these microspheres and taken up by surrounding tissue cells (e.g. endothelial cells).

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

In situations in which the therapeutic is a gene, a gene delivery system can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter or by stereotactic injection. A therapeutic gene, such as a gene encoding an antisense RNA or a ribozyme can be delivered in a gene therapy construct by electroporation using techniques known in the art.

A gene therapy preparation can consist essentially of a gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration.

The present invention is further illustrated by the following examples, which should not be construed as limiting in any way. The contents of all cited references (including literature references, issued patents, published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Materials and Methods

Animals

SP-C (−/−) mice were generated by targeted gene inactivation as previously described [11]. Generally, a 129/J mouse genomic library was screened to identify genomic clones of the SP-C gene homologous to the 129 derived ES cells. A 2.1-kb BamHI fragment containing exons 2-6 of the SP-C gene was used for modification of the gene. Sequence encoding the hydrophobic polyvaline domain of the SP-C peptide was interrupted by insertional mutagenesis with a 1.6-kb pGKneo gene cassette. This insertion provided positive selection for targeted cells by growth in the neomycin analogue G418. The 2.1-kb SP-C plasmid was digested with ApaLI, which cuts at a unique ApaLI site located in the SP-C polyvaline domain. The ApaLI linker pGKneoBPA cassette was ligated into the SP-C ApaLI site. A 1.3-kb PstI-to-BamHI fragment spanning exon 1 and the 5′ flanking DNA was ligated to the 5′ BamHI site of the 2.1-kb Bam-pGKneoBPA fragment. The targeting construct was modified further by cloning the herpes simplex virus thymidine kinase gene into the 5′ SphI site to provide gancyclovir selection against nonhomologous integration of the construct.

The D3R strain of ES cells was electroporated with the purified SP-C-targeting construct DNA and selected as described (15). ES cell DNA was digested with Bsu36I and analyzed with a probe outside of the targeting construct sequence. The probe was a 457-bp SphI-PstI fragment adjacent to the 5′ limit of the targeting construct. Positive clones were confirmed by genomic Southern blot of multiple restriction enzyme digests.

ES cell clones carrying a targeted SP-C allele were microinjected into C57/B16 blastocysts and implanted into host mice. Chimeric offspring were identified by mosaic Agouti coat color and bred to NIH Swiss black (Tac:N:NIHSBCfBr from Taconic Farms) females. Agouti offspring were screened for germ-line transmission of the targeted SP-C allele by genomic Southern blot analysis of BglII- and SphI-digested tail DNA. The 457 bp 5′ Sph-Pst fragment was used as a probe. The F1 offspring heterozygous for the SP-C mutation (+/−) were bred to establish a colony of SP-C (+/+), SP-C (+/−), and SP-C (−/−) mice. All mice were maintained in a pathogen-free barrier containment facility with filtered air, water, and autoclaved food.

Chimeric founder mice were bred to 129/Sv mice, Taconic (Germantown, N.Y.). Offspring were screened for transmission of the targeted SP-C allele by genomic Southern blot analysis. Animals positive for the targeted allele were bred to establish 129/Sv mice that were homozygous for the targeted SP-C allele. Mice were maintained in a barrier containment facility. All animals were handled under aseptic condition and caged in sterilized units with filtered air, water, and autoclaved food. Sentinel mice from this room were negative for common viral, bacterial or parasitic pathogens. At 12 months of age, lung homogenates prepared under aseptic conditions from SP-C (−/−) and wild type littermates did not contain bacteria or fungus. Serology for 23 mouse viral pathogens was negative.

Morphological Analysis

Mice were killed by intraperitoneal injection of a mixture of ketamine, xylazine, and acepromazine. Lungs were inflated by intratracheal instillation of 4% paraformaldehyde at 25 cm H₂O of pressure. After overnight fixation, the tissue was processed through conventional paraffin embedding. Six micron tissue sections were stained with hematoxylin-eosin, Mason's trichrome stain, or orcein stain. Immunohistochemical staining was performed for MAC-3, MUC5A/C, CCSP, SP-B, TTF-1, and α-SMA using biotinylated primary or secondary antibodies and avidin-biotin peroxidase (Vector Elite ABC Kit, Vector Laboratories, Inc., Burlingame, Calif.) or streptavidin (Zymed Laboratories, Burlingame, Calif.) using methods previously described [13]. Electronmicroscopy was performed on lung tissue obtained from 9 month old SP-C (−/−) and age matched controls after fixation in glutaraldehyde as previously described [14].

Phospholipid and Surfactant Proteins

Fifteen month old mice (n=5/group) were anesthetized with pentobarbital sodium (100 mg/kg ip) and killed by exsanguination. Trachea was cannulated and five 1 ml aliquots of 0.9% NaCl were flushed into the lungs and withdrawn by syringe three times for each aliquot. The lavaged lung tissue was removed and homogenized in 2 ml of 0.9% NaCl. Saturated phosphatidylcholine (Sat PC) in lipid extracts of bronchoalveolar lavage fluid (BALF) and lung tissue were isolated with osmium tetroxide [15] followed by phosphorus measurement [16], as previously described [11]. For phospholipid composition analyses, extracted lipid of lung tissue after BAL were used for two-dimensional thin-layer chromatography [17]. The spots were visualized with iodine vapor, scraped, and assayed for phosphorus content. Surfactant proteins in BALF were analyzed by Western blot after SDS-PAGE [11,18].

Cytokine Measurements

Concentrations of TNF-α, IL-I1β, IL-13, and IL-6 were measured in BALF and in whole lung homogenates post-lavage. Five animals of each genotype were assessed. ELISA kits were used according to manufacturer's instructions (R and D Systems, Minneapolis, Minn.).

MMP Activity

Matrix metalloproteinase (MMP-2 and MMP-9) activity was measured in macrophage conditioned media collected from 12 month old SP-C (−/−) or SP-C (+/+) 129/Sv mice, as previously described [19]. Macrophages were isolated by sequential lung lavage with 1 ml of PBS. Lavages were pooled and placed in culture at 5×105 cells per well of a 24 well tissue culture dish for 24 hours in serum free RPMI media supplemented with 1% Nutriodoma (Boehringer Mannheim, Indianapolis, Ind.) and 1% antibiotics. Proteinases from the conditioned media were concentrated by incubation of 100 μl media with 15 μl of gelatin-Sepharose 4-B beads (Amersham Pharmacia, Arlington Heights, Ill.) for three hours at 4° C. The beads were pelleted by gentle centrifugation, washed with PBS and the proteinases eluted by incubation of the beads in Laemmli sample buffer without BME for one hour at 37° C. Samples were directly analyzed by electrophoresis under nonreducing conditions into 10% Zymogram gelatin gels (NOVEX, San Diego, Calif.). Gels were washed twice with 2.5% Triton X-100 (15 minutes each) and incubated for 16 hours in a developing buffer (50 mM Tris pH 7.5, 200 mM NaCl, 5 mM CaCl2). Gels were then stained in 0.5% (wt/vol) Coomassie blue in 50% methanol, 10% acetic acid followed by partial destaining to reveal the clear bands of protease activity.

Lung Mechanics

Resistance and elastic forces were measured in airways and/or lung parenchyma of 15 month old wild type and SP-C (−/−) mice (n=5/group). Mice were anesthetized with 0.1 ml/10 g body weight of a mixture (ip) containing 40 mg/ml ketamine and 2 mg/ml xylazine. Mice were tracheostomized and respiratory impedance was measured by using the forced oscillation technique (0.25-20 Hz) delivered by computerized flexiVent (SCIREQ, Montreal, Canada) [20]. Estimated total lung compliance, airway resistance, airway elastance, tissue damping and tissue elastance for mice at 2 cm H₂O PEEP were obtained by fitting a model to each impedance spectrum. Hysteresivity was calculated as the ratio of tissue damping to tissue elastance. With this system, the calibration procedure removed the impedance of the equipment and tracheal tube.

Pressure-volume relationships were studied in 10-12 month old wild type and SP-C (−/−) mice (n=5/group). Mice were anesthetized with pentobarbital sodium (100 mg/kg ip) and placed in a box containing 100% O₂ to ensure complete collapse of the alveoli by O2 absorption. After the mice were killed by exsanguination, the cannula was inserted into trachea, connected to a pressure sensor (Mouse Pulmonary Testing System, TSS, Cincinnati, Ohio) and lung volume per kilogram body weight was determined at intervals of 5 cm H₂O during inflation and deflation [21].

Results

SP-C (−/−) 129/Sv Congenic Mice

SP-C (+/−) chimeric founders generated from 129/Sv embryonic stem cells and were bred to 129/Sv mice. Since only ES cell derived sperm transmit the SP-C mutation from the chimeric male founder, SP-C (−/−) offspring were produced entirely from 129/Sv germ cells. Thus, the SP-C (−/−) offspring represent an inbred strain. Poor health and reduced fecundity were noted in SP-C (−/−) mice by 2 months of age. Few litters were produced by animals older than 6 months of age. Poor grooming, and conjunctivitis were noted in all SP-C (−/−) 129/Sv animals beyond 6 months of age. Deterioration of coat condition was observed in most SP-C (−/−) mice after 2 months of age. The average body weight of 12-13 month old SP-C (−/−) mice was reduced by 24% (25.7 g±3.2, n=7 vs 33.5 g±3.0, n=7) compared to controls. In these older SP-C (−/−) mice, relative heart weight was increased as determined by heart/body weight ratios. Ratios were increased by 30% with the right ventricle being more enlarged than the left 0.00565±0.00026, m±SD, n=10 (SP-C −/−) vs 0.00431±0.00033, m±SD, n=7 (SP-C +/+), p<0.007.

Morphological Changes in the Lungs of SP-C (−/−) Mice

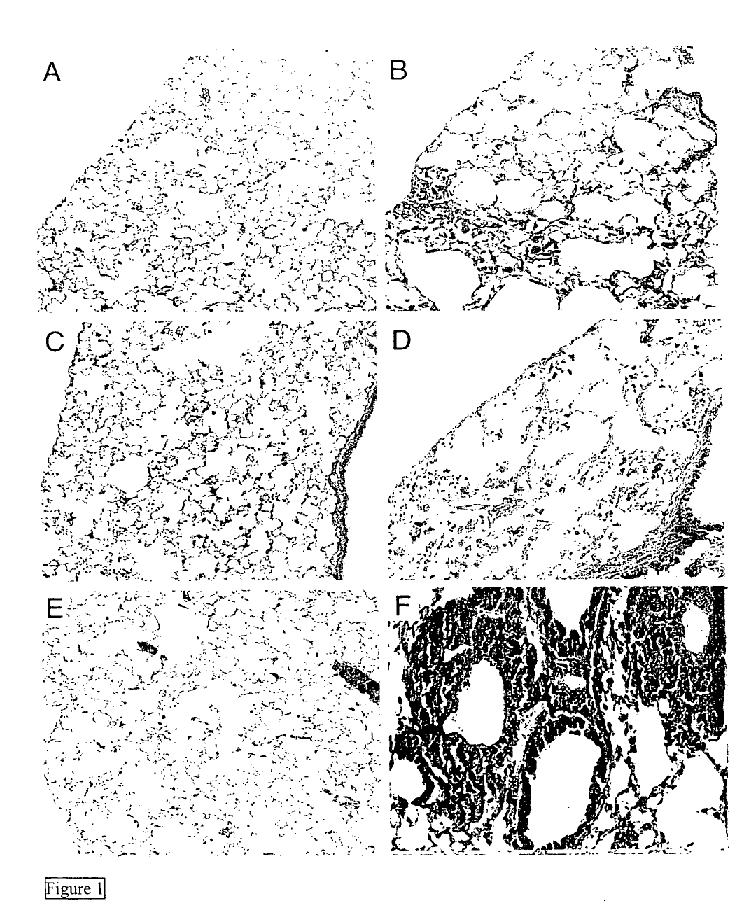

While lung structure of SP-C (−/−) mice was normal at birth (data not shown), enlargement of alveoli was observed by 2 months of age and thereafter, consistent with the development of emphysema, FIG. 1. Alveolar septation was irregular with absent or shortened alveolar septal tips observed throughout the lung parenchyma. Multifocal cellular infiltrates that generally consisted of alveolar macrophages and other mononuclear cells were detected, FIG. 1B. In lungs from 6 month old mice, consolidated parenchymal infiltrates were commonly observed. Extensive regions of type II cell hyperplasia and interstitial thickening were observed in the lung parenchyma. The extent and severity of parenchymal abnormalities and cellular infiltrates increased with age, often resulting in regions with complete obliteration of some alveolar spaces at 12 months of age. Areas with epithelial cell hyperplasia, interstitial thickening, and fibrosis were observed in alveoli and airways. Extensive perivascular, and peribronchiolar monocytic infiltrates were detected in the most severely affected animals, FIG. 1F.

Alveolar Remodeling

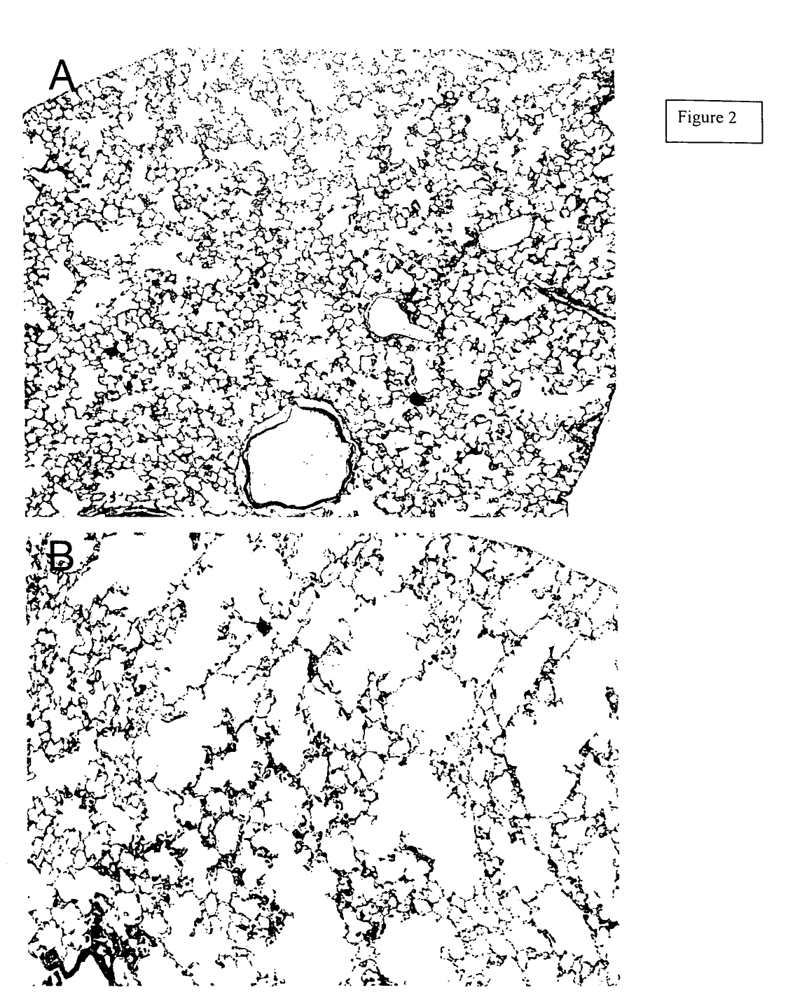

Trichrome staining demonstrated regions of fibrosis in the lung parenchyma, pleural surfaces and at perivascular and peribronchiolar sites, FIG. 2. In some regions, collagen deposition was distributed in extended web-like configurations throughout the lung parenchyma. Extensive α-SMA staining, indicating myofibroblast transformation, was observed throughout the alveoli of SP-C (−/−) mice. The intensity and extent of α-SMA staining was in general, increased with age, but variable within lung sections and among littermates, FIG. 2. Loss of the network of alveolar elastin fibers detected with orcein stain was observed in areas of alveolar disruption in the SP-C (−/−) mice, FIG. 2. Regions with reduced orcein staining colocalized with sites of increased trichrome staining, supporting the concept that the severity of alveolar remodeling was correlated with pulmonary fibrosis in SP-C deficient mice.

Electronmicroscopic Findings

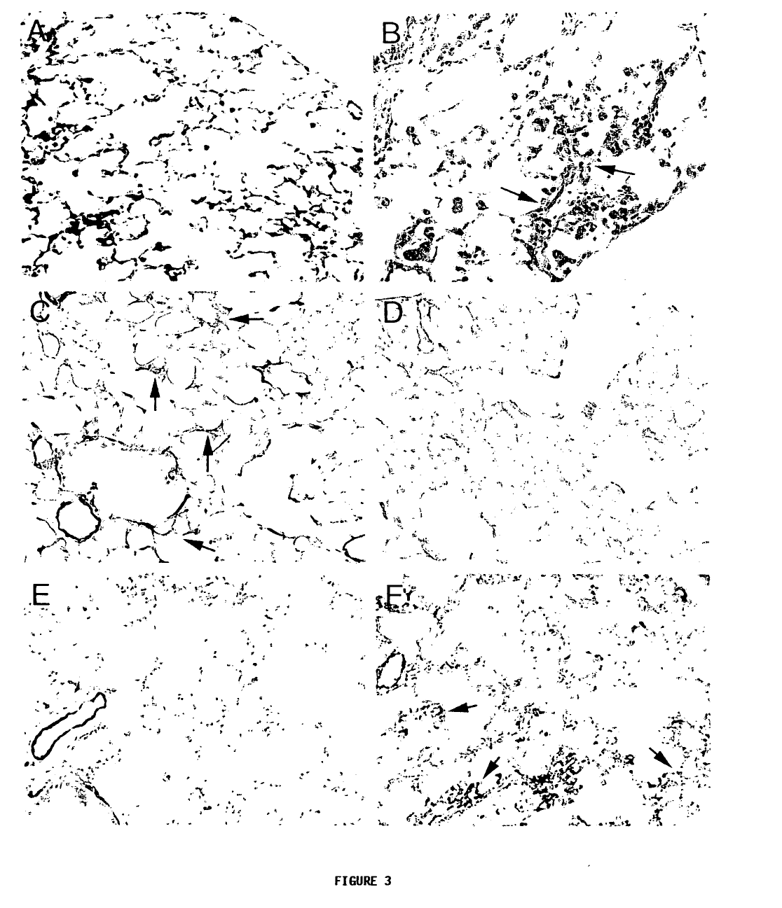

At the electronmicroscopic level, alveoli of the SP-C (−/−) mice were often thickened and lined by hyperplastic type II epithelial cells, FIG. 3A. Increased numbers of cuboidal cells were observed lining alveolar surfaces, and type II cells contained excessive numbers of lamellar bodies. Capillary walls were thickened or obliterated by surrounding stroma and collagen. Bronchi and bronchioles were lined by a highly atypical columnar epithelia. Conducting airways were lined by non-ciliated columnar epithelial cells that contained numerous atypical electron dense organelles, consistent with the atypical mitochondria characteristic of Clara cells [22]. Type II cells were hypertrophic, containing increased numbers of lamellar bodies and lipid inclusions. In the alveolus, basement membranes were thickened, containing numerous collagen fibrils. Many capillary lumena were obliterated, and regions of fibrosis were readily discerned. Basement membranes and endothelial surfaces of larger vessels were disrupted. Abnormal alveolar macrophages contained large accumulations of surfactant like material with structural features of tubular myelin and lamellar bodies, FIG. 3.

Macrophage Morphology and Abnormal Lipid Accumulations

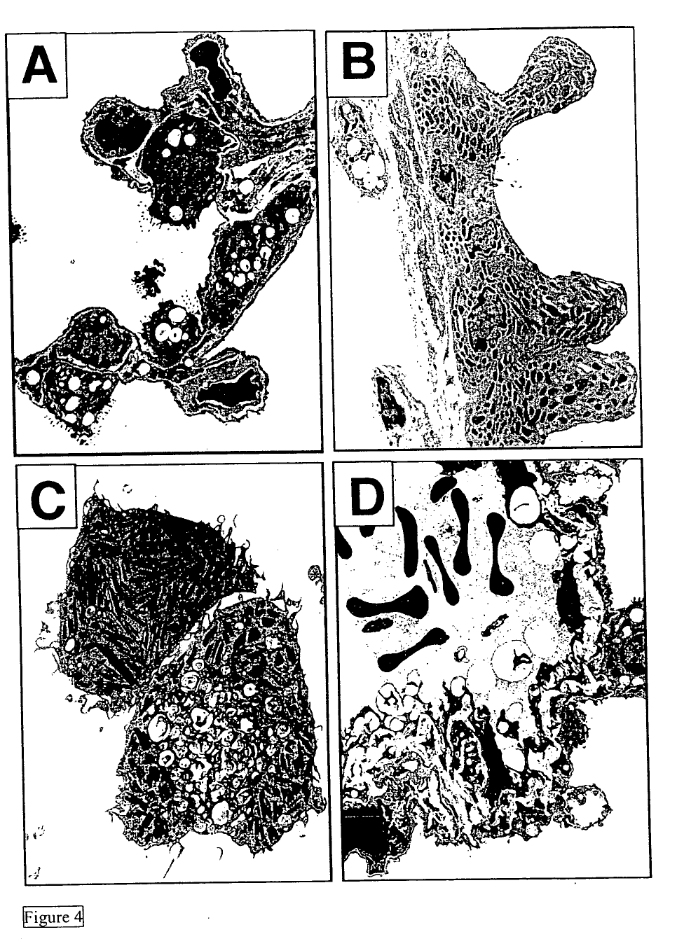

Subsets of mononuclear cells in the alveolar spaces of SP-C (−/−) mice stained intensely with the MAC3 antibody, an alveolar macrophage cell marker, FIG. 4B. Abnormal intracellular lipid inclusions were observed in alveolar macrophages, FIG. 4D. Likewise, lipid accumulations were also noted in the hyperplastic type II epithelial cells lining residual alveoli, FIG. 4D. At the ultrastructural level, the atypical alveolar macrophages contained abundant surfactant components including lamellar bodies and tubular myelin, extracellular forms of pulmonary surfactant, FIG. 3. Other macrophages contained numerous cytoplasmic crystals consistent with those formed by Ym1, a mammalian lectin [23]. Mass spectroscopic analysis confirmed the presence of increased Ym1 in the BALF (data not shown). Accumulation of the intracellular crystals and lipids was not detected in alveolar macrophages from control 129/Sv maintained in this barrier facility. In BALF from 6 month old SP-C (−/−) mice, the number of alveolar macrophages was increased 4.4 fold, 9021±1017 vs 2039±497, (n=5) in SP-C (−/−) vs SP-C (+/+), respectively. The percentage of lymphocytes was not altered. Changes in polymorphonuclear cells and eosinophils were not observed.

Epithelial Cell Dysplasia

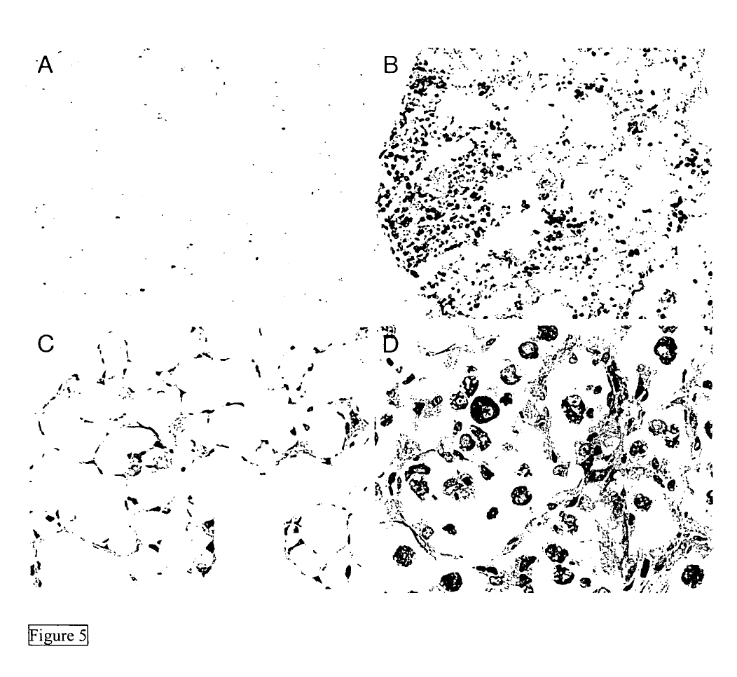

Pronounced changes in conducting airway epithelial cell morphology were observed in SP-C (−/−) mice, FIG. 5. Epithelial cell dysplasia was readily apparent at 6 to 12 months of age, the conducting airways being lined by hyperplastic, pseudostratified columnar epithelium, FIGS. 1F, 5B. While MUC5A/C staining cells were rarely seen in wild type mice, MUC5A/C positive cells lined most of the conducting airways of the SP-C (−/−) mice, FIG. 5D. MUC5A/C staining of conducting airways was generally extensive, however heterogeneity in the pattern of staining occurred. Immunostaining for Clara cell secretory protein (CCSP) and proSP-B was detected, but the extent and intensity of staining was decreased in severely affected conducting airways in SP-C (−/−) mice, also consistent with epithelial cell dysplasia (data not shown). In the alveoli, septal thickening and dense monocytic infiltration were noted in the areas of extensive epithelial hyperplasia. However, in some areas with severe airspace remodeling, some alveoli lacked type II cells. In those lesions, web-like strands of squamous cells formed alveoli that were devoid of capillaries.

Pulmonary Mechanics

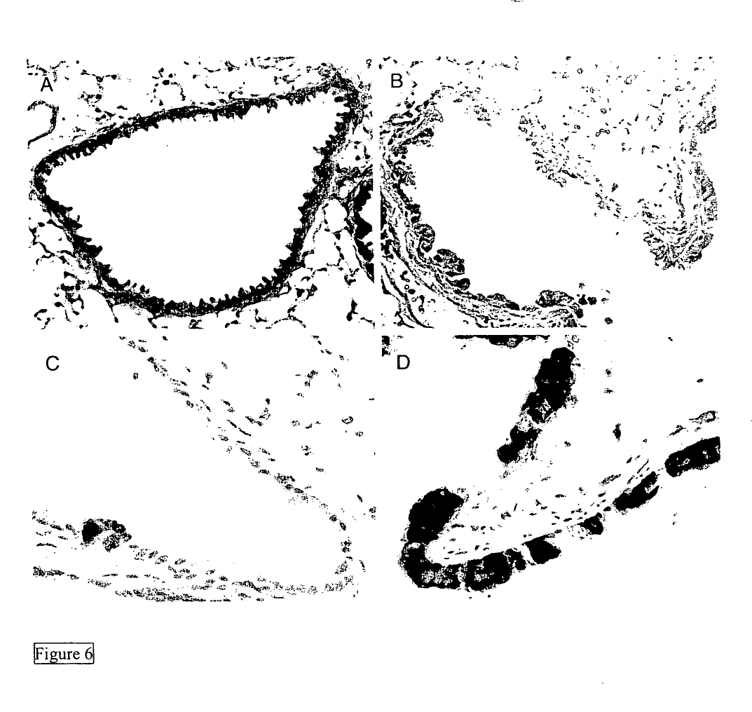

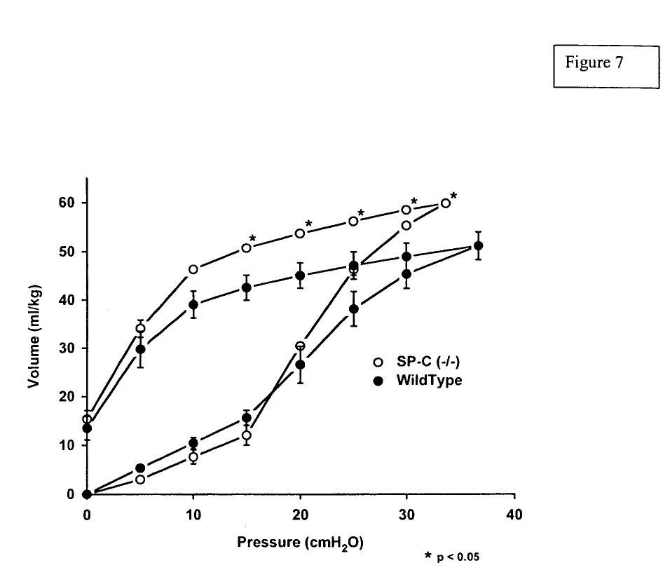

At higher pressures on the deflation limb of pressure-volume curves, lung volumes were significantly increased in SP-C (−/−) compared to wild type mice (FIG. 6), consistent with the emphysema observed histologically, FIG. 1. At lower pressures, lung volumes were normal and residual lung volumes were maintained at 0 pressure, consistent with normal surfactant function. Similarly, there were no significant differences between SP-C (−/−) and control mice in dynamic lung compliance obtained with ventilation volumes of 7 ml/kg, Table II. While airway and tissue elastance was unaltered, both airway resistance and tissue damping was significantly increased in SP-C (−/−) mice (p<0.05). Hysteresivity was significantly increased in the SP-C (−/−) mice (p<0.01). These findings are consistent with the observed emphysema and with maintenance of surfactant function. Surfactant Composition Tissue and total surfactant phospholipid pool sizes were increased approximately 2-fold in SP-C (−/−) mice, FIG. 7. The composition of lipids in lung tissue after BAL was unchanged, Table I. SP-A, SP-B, and SP-D were estimated by Western blot analysis of BALF. While surfactant protein B levels were unaltered, SP-A and SP-D were significantly increased in SP-C (−/−) mice.

TABLE I
Phospholipid Content in Lung Tissue

| | SP-C (+/+) | SP-C (−/−) |
|---|---|---|
| | μmol/kg | μmol/kg |
| SM | 6.8 ± 0.6 | 5.9 ± 1.0 |
| Lyso PC 1 | 8.4 ± 1.0 | 21.5 ± 3.3 |
| PC | 70.0 ± 3.1 | 82.5 ± 12.4 |
| PI | 2.8 ± 0.2 | 2.5 ± 0.4 |
| PS | 5.3 ± 1.2 | 6.1 ± 0.6 |
| PE | 22.9 ± 1.5 | 25.8 ± 4.0 |
| PG | 3.3 ± 0.8 | 4.2 ± 0.5 |

Values are mean ± SE, n = 5 per group.
SM: Sphingomyelin;
PS: Phosphatidylserine;
PC: Phosphatidylcholine;
PE: Phosphatidylethanolamine;
PI: Phosphatidylinositol;
PG: Phosphatidylglycerol

TABLE II
Mechanical Parameters of the Lung obtained by
using Forced Oscillation Technique

| | SP-C (+/+) | SP-C (−/−) |
|---|---|---|
| Compliance (ml/cmH₂O · kg) | 1.45 ± 0.11 | 0.69 ± 0.01 |
| Airway Resistance (cmH₂O · s/ml) | 20.2 ± 0.5 | 2.38 ± 0.03 |
| Airway Elastance (cmH₂O/ml) | 19.3 ± 0.7 | 0.123 ± 0.006 |
| Tissue Damping (cmH₂O/ml) | 1.54 ± 0.10 | 0.80 ± 0.04* |
| Tissue Elastance (cmH₂O/ml) | 20.1 ± 0.8 | 3.34 ± 0.21* |
| Hysteresivity | 20.0 ± 1.0 | 0.167 ± 0.008* |

Values are mean ± SE.
*p < 0.05 as assessed by two tailed Student t-test,
n = 5 per group.

Cytokine and Metalloproteinase Expression

Concentrations of proinflammatory cytokines were determined in BALF and lung homogenates from 6 month old mice. TNF-α, IL-6, MIP-2, and IL-13 were not altered in the SP-C (−/−) mice. The supernatants of cultured alveolar macrophages from SP-C (−/−) and (+/+) were tested for MMP activity by SDS/PAGE zymography at one year of age. Gelatinase activity was readily detectable in the conditioned media from SP-C (−/−) macrophages but was undetectable in media from control macrophages (SP-C +/+). Proteinase bands migrated at approximately 72 kDa and 92 kDa, consistent with MMP-2 and MMP-9, respectively (data not shown). In addition, MMP-12 mRNA was increased 3.58 fold in lung RNA from SP-C (−/−) compared to wild type mice. The elevated expression of MMP activity may contribute to alveolar remodeling seen in the SP-C (−/−) mice.

Discussion

A severe pulmonary disorder characterized by emphysema, epithelial cell dysplasia, monocytic cell infiltration, pulmonary fibrosis and abnormal lipid accumulations, was caused by targeted deletion of the SP-C gene in a congenic strain of SP-C (−/−)/129/Sv mice. Heterogeneous pulmonary lesions contained 1) thickened, fibrotic alveolar walls that stained for α-smooth muscle actin, 2) extensive monocytic infiltrates and increased expression of metalloproteinases, 3) regions of severe emphysema with septal thinning and degeneration of pulmonary capillaries, 4) epithelial cell dysplasia and MUC5A/C expression in conducting airways, and 5) accumulation of intracellular lipids in various cell types. Pathologic findings in the SP-C (−/−) mice were consistent with, but not identical to, those seen in lungs from patients with various conditions termed idiopathic interstitial pneumonitis (IIP). Thus, lack of SP-C or proSP-C can be directly linked to the pathogenesis of interstitial lung disease in mice.

A mutation in the SP-C gene was recently associated with familial interstitial pneumonitis that was inherited as an autosomal dominant affect [8,9]. In a sibship with mutation c460+1G→A, resulting in an exon 4 deletion of the proSP-C peptide, misprocessed proSP-C accumulated within type II epithelial cells; tissue and lung lavage material lacked the active SP-C peptide [8]. Similarly, a single base pair substitution (L188Q) altered subcellular localization of proSP-C in an extended family with IIP [9]. Therefore, it has been unclear whether the severe pulmonary disease in these patients results from the lack of SP-C, or to abnormal accumulations of misfolded mutant SP-C or proSP-C proteins. The present studies demonstrate that the lack of SP-C per se, can recapitulate many of the pathologic findings consistent with various forms of adult and childhood interstitial pneumonitis.

While the absence of proSP-C and/or SP-C caused severe lung disease in the mouse, the molecular pathogenesis of this disorder remains unclear. At the light microscopic level, lung structure in the SP-C (−/−) mice was normal at E19.5 and postnatal day 1 (data not shown). Abnormalities seen in lung structure increased with advancing age, suggesting that emphysema and remodeling do not arise from abnormalities in lung morphogenesis, but from ongoing injury and repair processes. The expression of various pro-inflammatory cytokines that have been previously associated with emphysema and inflammation were not altered in the SP-C (−/−) mice. There was no change in neutrophil number, and there was no evidence of viral or bacterial infection in SP-C (−/−) mice. These findings suggest that the remodeling and inflammation are caused by cellular abnormalities intrinsic to the lung, and dependent upon the functions of SP-C or perhaps the result of selective degradation of extracellular matrix by MMPs elaborated by the macrophages rather than to susceptibility to pathogens. MMP-9 and MMP-2 production by alveolar macrophages, and MMP-12 mRNA levels were increased and therefore may play a role in the pathogenesis of the lung disease in the SP-C (−/−) mice. Increased MMP-2, MMP-9, and MMP-12 expression was previously associated with emphysema in SP-D gene targeted mice [24].

While pro-inflammatory cytokines were not increased in the lungs of the SP-C (−/−) mice, the lungs were infiltrated with atypical alveolar macrophages containing numerous lipid inclusions and Ym1 crystals [23]. The numbers of the abnormal macrophages were increased 4-5 fold compared to control. Cellular infiltration was associated with alveolar thickening and fibrosis. The myofibroblast transformation and collagen deposition seen at the ultrastructural level were consistent with increased α-SMA staining seen throughout the alveolar walls of the SP-C (−/−) mice. Paradoxically, marked epithelial cell dysplasia was observed in conducting airways in the SP-C (−/−) mice, in spite of the fact that proSP-C is not expressed in these cells in wild type mice. Furthermore, high levels of expression of MUC5A/C were observed in the conducting airways at sites in which SP-C mRNA and protein are not normally expressed. MUC5A/C is normally expressed at low levels in the conducting airways of mice, but is readily induced by inflammation or inflammatory cytokines, being increased by IL-4, IL-13, and allergens [25 for review]. These latter findings suggest that the lack of SP-C may influence gene expression outside the alveolus, implying that SP-C plays a role, directly or indirectly, in the conducting airways. However, it is unclear whether cellular abnormalities in the conducting airways of SP-C (−/−) mice are mediated directly by SP-C dependent signaling events or might be related to SP-C dependent modulation of surface forces or changes in mucociliary clearance in the absence of SP-C.

The finding that severe lung disease can be caused by either the expression of a dominantly inherited mutant proSP-C protein or the deletion of SP-C gene suggests several potential mechanisms by which SP-C may contribute to the pathogenesis of IIP. In the IIP patients described by Nogee et al. and Thomas et al. [8,9], the mutant proSP-C protein accumulated within type II cells, potentially creating cell injury related to the misfolding or misprocessing of the precursor protein. In support of this concept, Conkright et al. recently demonstrated that expression of an SP-C mutant protein caused lethal lung dysfunction in vivo [26]. However, the active SP-C peptide was absent in the SP-C (−/−) mice and in patients with IPF caused by this dominantly inherited SP-C mutation [8]. Thus, the lack of SP-C per se may be involved in the pathogenesis of IIP. Amin et al. recently described a sibship in which three individuals were severely affected by IIP, each of whom lacked detectable expression of either proSP-C or SP-C in alveolar lavage, in spite of the failure to find mutations in the coding region of SP-C [27]. Whether the selective lack of proSP-C or SP-C directly caused the disorder in these patients is unclear.

Do Abnormalities in Surfactant Function Contribute to IIP?

The present findings demonstrate that the lack of SP-C per se causes a syndrome with features of interstitial pneumonitis in mice. Since SP-C enhances surface properties of phospholipids in the airspace, it is possible that the lack of SP-C alters surfactant function in time leading to interstitial pneumonitis. However, lung phospholipid content was unaltered in SP-C (−/−) mice in the Swiss black strain [11], and was increased 2-fold in SP-C (−/−) mice in 129/Sv background. Surfactant phospholipid composition, structure of lamellar bodies and tubular myelin were generally preserved in both strains of SP-C (−/−) mice. Changes in lung mechanics and lung histology shown in a previous study of 8 week old Swiss black SP-C (−/−) mice were distinct from the present study. In SP-C (−/−) Swiss black mice, there was no evidence of inflammation or emphysema. Hysteresivity was decreased while tissue elastance and resistance were unaltered, findings consistent with a modest abnormality of in vitro surface activities of surfactant. In contrast, the SP-C (−/−) mice in the present study showed severe abnormalities in airway resistance, tissue damping, and hysteresivity was significantly increased, consistent with the extensive emphysema [28]. Furthermore, SP-B and surfactant phospholipid pool sizes were normal or increased, consistent with the observed preservation of surfactant function. The modestly increased levels of SP-A and SP-D in the SP-C (−/−) 1 29/Sv mice may reflect changes related to chronic lung inflammation. Thus, there is no evidence at present that surfactant deficiency accounts for the chronic lung disease in the SP-C (−/−) 129/Sv mice, but it remains possible that subtle differences in sheer forces not discernable in the present studies may contribute to the disruption of lung structure and function in the SP-C (−/−) mice. In vitro studies demonstrate that various growth factors, cytokines, and sheer stress can cause myofibroblast transformation of lung fibroblasts. Consistent with the increased α-SMA staining observed in the present study in mice, the extensive fibrosis and myofibroblast transformation is often seen in humans with IIP [10]. Collagen deposition and increased numbers of fibroblasts are also readily observed within the alveolar walls, similar to that seen in human patients with IIP. If lack of SP-C contributes to the pathogenesis of the pulmonary disease, therapy in which exogenous SP-C is administered might be considered for patients with IIP. On the other hand, if the disorder is caused by misrouting and abnormal accumulations of SP-C or mutant SP-C, the addition or increased expression of normal SP-C may actually contribute to the disorder.

Does SP-C Deficiency Cause a Lipid Storage Disease?

Surfactant lipids, lamellar bodies, and tubular myelin accumulated in the atypical macrophages, and prominent lipid droplets were observed in the abundant fibroblasts underlying type H cells in the lungs of SP-C (−/−) mice. These pathologic findings suggest the possibility that the absence of SP-C alters the catabolism of surfactant, or other cellular constituents, creating a storage disorder. In vitro studies have demonstrated that SP-C enhances surfactant lipid uptake by type II epithelial cells, functioning in a manner distinct from that of SP-A and SP-B, the latter serving to maintain large surfactant aggregates associated with the epithelial surfaces [29]. Thus SP-C may have both intracellular and extracellular roles in surfactant homeostasis.

Strain Influences the Pathologic Finding in the SP-C (−/−) Mice

The severe lung disease observed in the SP-C (−/−) mice in the 129/Sv strain contrasts sharply with the milder abnormalities seen in SP-C (−/−) mice when maintained in outbred Swiss black background. While the SP-C (−/−)/Swiss black mice do not have overt abnormalities in lung structure, these mice are susceptible to lung dysfunction when placed in hyperoxia and reduction of surfactant protein B [12]. The strong strain-dependent influence on the SP-C (−/−) phenotype and the heterogeneity of pulmonary lesions that vary in severity and time and place, are consistent with findings in patients with familial idiopathic fibrosis caused by mutations in the SP-C gene [30]. These syndromes are clinically and pathologically distinct from the emphysema associated with α1-antitrypsin deficiency. In IIP, clinical and pathologic findings vary greatly in these sibships, and multiple histopathological diagnoses have been made within the same family. While the nature of the SP-C mutations may influence the disorder, marked heterogeneity in severity, age of presentation, and time of progression of pulmonary disease characterizes this disorder, suggesting that environmental factors or other genes strongly influence its pathogenesis. The observed strain differences in the severity of lung disease caused by SP-C deficiency in the SP-C (−/−) mice, suggest that the phenotype associated with SP-C deficiency or SP-C mutations may be strongly influenced by genetic factors. While there is no evidence that infection complicated the interpretation of the present study, lung dysfunction in patients with IIP is exacerbated by infection.

Implications for Diagnosis and Therapy

The present study and recent human studies [8,30] provide perhaps the first association between gene mutations and idiopathic interstitial lung disease. Since the absence of SP-C caused severe lung disease in the SP-C (−/−) mice, it is also possible that deficiency of SP-C, whether genetic or secondary to injury, may contribute to acute and chronic lung disease. The association between mutations in SP-C with IIP in humans, makes feasible genetic testing for the risk of the disease. Likewise, histologic diagnosis of the various pathologies caused by mutations in the SP-C gene can be made by immunohistochemistry. Detection of mutant SP-C genes or the presence or absence of SP-C from BALF may provide diagnostic insights into the role of SP-C in patients with complex lung diseases. Finally, it is unclear whether human BP is caused by 1) the absence of SP-C and proSP-C, 2) misfolding and misrouting of either the SP-C proprotein or the active SP-C peptide, or 3) altered routing, processing or degradation of other cellular components whose homeostasis is dependent upon proSP-C and/or SP-C. If protein misfolding in type II or other lung cells contributes to the pathogenesis of lung disease, the misfolding of proteins other than SP-C may be considered in the pathogenesis of interstitial lung disease. Clarification of cellular and molecular mechanisms causing interstitial lung disease related to abnormalities in SP-C may provide a conceptual basis for the development of new therapies for IIP.

References
1. Weaver, T. E., and Conkright, J. J. 2001. Functions of surfactant proteins B and C. Ann. Rev. Physiol. 63:555-578.
2. Johansson, J. 1998. Structure and properties of surfactant protein C. Biochim Biophys Acta 1408:161-172.
3. Morrow, M. R., Taneva, S., Simatos, G. A., Allwood, L. A., and Keough, K. M. 1993. 2H NMR studies of the effect of pulmonary surfactant SP-C on the 1,2-dipalmitoyl-snglycero-3-phosphocholine headgroup: a model for transbilayer peptides in surfactant and biological membranes. Biochemistry 32:11338-11344.
4. Horowitz, A. D., Elledge, B., Whitsett, J. A., and Baatz, J. E. 1992. Effects of lung surfactant proteolipid SP-C on the organization of model membrane lipids: a fluorescence study. Biochim. Biophys. Acta 1107:44-54.
5. Johansson, J., Szyperski, T., and Wuthrich, K. 1995. Pulmonary surfactant-associated polypeptide SP-C in lipid micelles: CD studies of intact SP-C and NMR secondary structure determination of depalmitoyl-SP-C(1-17). FEBS Lett. 362:261-265.
6. Whitsett, J. A., Ohning, B. L., Ross, G., Meuth, J., Weaver, T., Holm, B. A., Shapiro, D. L., and Notter, R. H. 1986. Hydrophobic surfactant-associated protein in whole lung surfactant and its importance for biophysical activity in lung surfactant extracts used for replacement therapy. Pediatr. Res. 20:460-467.
7. Hawgood, S., Ogawa, A., Yukitake, K., Schlueter, M., Brown, C., White, T., Buckley, D., Lesikar, D., and Benson, B. 1996. Lung function in premature rabbits treated with recombinant human surfactant protein-C. Am. J. Respir. Crit. Care Med. 154:484-490.
8. Nogee, L. M., Dunbar, A. E., Wert, S. E., Askin, F., Hamvas, A., and Whitsett, J. A. 2001. A mutation in the surfactant protein C gene associated with familial interstitial lung disease. N. Engl. J. Med. 344:573-579.
9. Thomas, A. Q., Lane, K., Phillips, J. III, Prince, M., Markin, C., Speer, M., Schwartz, D. A., Gaddipati, R., Marney, A., Johnson, J., et al. 2002. Heterozygosity for a surfactant protein C gene mutation associated with usual interstitial pneumonitis and cellular nonspecific interstitial pneumonitis in one kindred. Am. J. Respir. Crit. Care Med. 165:1322-1328.
10. Katzenstein, A. L., and Myers, J. L. 1998. Idiopathic pulmonary fibrosis: clinical relevance of pathologic classification. Am. J. Respir. Crit. CareMed. 157:1301-1315.
11. Glasser, S. W., Burhans, M. S., Korfhagen, T. R., Na, C-L., Sly, P. D., Ross, G. F., Ikegami, M., and Whitsett, J. A. 2001. Altered stability of pulmonary surfactant in SP-C-deficient mice. Proc. Natl. Acad. Sci. USA 98:6366-6371.
12. Ikegami, M., Weaver, T. E., Conkright, J .J., Sly, P. D., Ross, G. F., Whitsett, J. A., and Glasser, S. W. 2002. Deficiency of SP-B reveals protective role of SP-C during oxygen injury. J. Appl. Physiol. 92:519-526.
13. Wert, S. E., Dey, C. R., Blair, P. A., Kimura, S., and Whitsett, J. A. 2002. Increased expression of thyroid transcription factor-1 (TTF-1) in respiratory epithelial cells inhibits alveolarization and causes pulmonary inflammation. Dev. Biol. 242:75-87.
14. Clark, J. C., Tichelaar, J. W., Wert, S. E., Itoh, N., Perl, A. K., Stahlman, M. T., and Whitsett, J. A. 2001. FGF-10 disrupts lung morphogenesis and causes pulmonary adenomas in vivo. Am. J. Physiol. 280:L705-L715.
15. Mason, R. J., Nellenbogen, J., and Clements, J. A. 1976. Isolation of disaturated phosphatidlycholine with osmium tetroxide. J. Lipid Res. 17:281-284.
16. Bartlett, G. R. 1959. Phosphorus assay in column chromatography. J. Biol. Chem. 234:466-468.
17. Jobe, A. H., Kirkpatrick, E., and Gluck, L. 1978. Labeling of phospholipids in the surfactant and subcellular fractions of rabbit lung. J. Biol. Chem. 253:3810-3816.
18. Ikegami, M., Whitsett, J. A., Jobe, A., Ross, G., Fisher, J., and Korfhagen, T. 2000. Surfactant metabolism in SP-D gene-targeted mice. Am. J. Physiol. 279:L468-L476.
19. Yoshida, M., Korfhagen, T. R., and Whitsett, J. A. 2001. Surfactant protein D regulates NF-κB and matrix metalloproteinase production in alveolar macrophages via oxidantsensitive pathways. J. Immunol. 166:7514-7519.
20. Schuessler, T. F., and Bates, J. H. 1995. A computer controlled research ventilator for small animals: design and evaluation. IEEE Trans Biomed Eng. 42:860-866.
21. Ikegami, M., Jobe, A. H., Whitsett, J., and Korfhagen, T. 2000. Tolerance of SP-A deficient mice to hyperoxia or exercise. J. Appl. Physiol. 89:644-648.
22. Cross, P. C., and Mercer, K. L. 1993. Respiratory System. In Cell and tissue ultrastructure: a functional perspective. W. H. Freeman/New York, 314-315.
23. Chang, N. C., Hung, S. I., Hwa, K. Y., Kato, I., Chen, J. E., Liu, C. H., and Chang, A. C. 2001. A macrophage protein, Ym1, transiently expressed during inflammation is a novel mammalian lectin. J. Bol. Chem. 276:17497-17506.
24. Wert, S. E., Yoshida, M., LeVine, A. M., Ikegami, M., Jones, T., Ross, G. F., Fisher, J. H., Korfhagen, T. R., and Whitsett, J. A. 2000. Increased metalloproteinase activity, oxidant production, and emphysema in surfactant protein D gene-inactivated mice. Proc. Natl. Acad. Sci. USA 97:5972-5977.
25. Rose, M. C., Nickola, T. J., and Voynow, J. A. 2001. Airway mucus obstruction: mucin glycoprotiens, MUC gene regulation and goblet cell hyperplasia. Am. J. Respir. Cell Mol. Biol. 25:533-537.
26. Conkright, J. J., Na, C.-L., and Weaver, T. E. 2002. Overexpression of SP-C mature SP-C peptide causes neonatal lethality in transgenic mice. Am. J. Respir. Cell Mol. Biol. 26:85-90.
27. Amin, R. S., Wert, S. E., Baughman, R. P., Tomashefski, J. F., Nogee, L. M., Brody, A. S., Hull, W. M., and Whitsett, J. A. 2001. Surfactant protein deficiency in familial interstitial lung disease. J. Pediatr. 139:85-92.
28. Pillow, J. J., Korfhagen, T. R., Ikegami, M., and Sly, P. D. 2001. Overexpression of TGFalpha increases lung tissue hysteresvity in transgenic mice. J. Appl. Physiol. 91:2730-2734.
29. Horowitz, A. D., Moussavian, B., and Whitsett, J. A. 1996. Roles of SP-A, SP-B and SP-C in modulation of lipid uptake by pulmonary epithelial cells in vitro. Am J. Physiol. (Lung Cell. Mol. Physiol.) 270:L69-L79.
30. Nogee, L. M., Dunbar, A. E., Wert, S., Askin, F., Hamvas, A., and Whitsett, J. A. 2002. Mutations in the surfactant protein C gene associated with interstitial lung disease. Chest 121:20S-21 S.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A transgenic non-human mammal, wherein the mammal carries a targeted disruption in the coding sequence of an endogenous surfactant protein C (SP-C) gene and wherein the targeted disruption inhibits production of wild-type surfactant protein C so that the phenotype of the mammal is characterized by a pulmonary disorder condition consistent with changes in humans with familial SP-C deficiency.

2. The transgenic mammal of claim 1, wherein the mammal develops a severe progressive pulmonary disorder with histologic features consistent with interstitial pneumonitis.

3. The transgenic mammal of claim 2, wherein the phenotype of the mammal comprises at least one phenotype selected from the group consisting of emphysema, monocytic infiltrates, fibrosis, epithelial cell dysplasia, and atypical accumulations of intracellular lipids in type II epithelial cells and alveolar macrophages.

4. The transgenic mammal of claim 3, wherein the pulmonary disorder condition is consistent with changes in humans with familial SP-C deficiency.

5. The transgenic mammal of claim 4, wherein the mammal is heterozygous for the disruption in the surfactant protein C gene.

6. The transgenic mammal of claim 4, wherein the mammal is homozygous for the targeted disruption in the surfactant protein C gene.

7. The transgenic mammal of claim 4, wherein the phenotype includes damage to the lung tissue.

8. The transgenic mammal of claim 4, wherein the mammal is a mouse.

9. The transgenic mouse of claim 8, wherein the mouse is derived from a 1 29/Sv mouse line.

10. The transgenic mouse of claim 8, wherein the disruption is created by insertional disruption of exon 2.

11. The transgenic mouse of claim 8, wherein the targeted disruption includes at least nucleotide position 1667 at the ApaL1 site in exon 2 of the wild type surfactant protein C gene.

12. The transgenic mammal of claim 4, wherein the disruption is created by a deletion of at least 50 consecutive nucleotides of coding sequence of the surfactant protein C gene.

13. A cell or cell line from a transgenic mouse, wherein the cell or cell line contains a targeted disruption in the coding sequence of an endogenous surfactant protein C (SP-C) gene.

14. The cell or cell line of claim 13, wherein the mouse is derived from a 129/Sv mouse line.

15. The cell or cell line of claim 13, wherein the disruption is created by insertional disruption of exon 2.

16. The cell or cell line of claim 15, wherein the disruption includes at least nucleotide position 1667 at the ApaL1 site in exon 2 of the wild type surfactant protein C gene.

17. The cell or cell line of claim 13, wherein the targeted disruption is created by a deletion of at least 50 consecutive nucleotides of coding sequence of the surfactant protein C gene.

18. The cell or cell line of claim 13, which is an undifferentiated cell.

19. The cell or cell line of claim 14, wherein the undifferentiated cell is selected from the group consisting of a stem cell, embryonic stem cell oocyte and embryonic cell.

20. A method of producing a mouse with a targeted disruption in a surfactant protein C (SP-C) gene, comprising the steps of:
a. creating a knockout construct comprising a portion of the SP-C gene with an internal portion of said SP-C gene replaced by a marker, wherein at least 50 consecutive nucleotides of SP-C gene coding sequence have been deleted;
b. transfecting said knockout construct into a population of embryonic stem cells and selecting a transfected ES cell which expresses said marker;
c. introducing said transfected ES cell into an embryo of an ancestor of said mouse;
d. allowing said embryo to develop to term to produce a chimeric mouse with the knockout construct in its germline;
e. breeding said chimeric mammal, to produce a heterozygous mouse with a targeted disruption in the SP-C gene.

21. An surfactant protein C knock-out construct, comprising a portion of an surfactant protein C (SP-C) gene, wherein an internal portion of said SP-C gene is replaced by a selectable marker and at least 50 consecutive nucleotides of SP-C gene coding sequence have been deleted.

22. The SP-C knockout construct of claim 21, wherein the selectable marker is a gene encoding a protein selected from the group consisting of thymidine kinase, neomycin phosphotransferase and hygromycin B phosphotransferase.

23. The SP-C knock-out construct of claim 21, wherein the marker is a neomycin resistance gene.

24. A method of testing an agent for effectiveness against a pulmonary condition, said method comprising:
a. obtaining a transgenic mouse that is homozygous for an surfactant protein C null allele wherein the transgenic mouse exhibits a phenotype selected from the group consisting of emphysema, monocytic infiltrates, fibrosis, epithelial cell dysplasia, and atypical accumulations of intracellular lipids in type II epithelial cells and alveolar macrophages, and
b. administering said agent to said transgenic animal;
wherein an agent that ameliorates said phenotype is selected as an agent that has effectiveness against said condition.

25. The method of claim 20, wherein the ancestor of said mouse is a 129/Sv mouse.

26. The method of claim 24, wherein the mouse is derived from a 129/Sv mouse line.

27. The method of claim 24, wherein the surfactant protein C null allele is created by a targeted disruption in the coding sequence of an endogenous surfactant protein C (SP-C) gene.

28. The method of claim 24, wherein the surfactant protein C null allele is created by insertional disruption of exon 2.

29. The method of claim 24, wherein the disruption includes at least nucleotide position 1667 at the ApaL1 site in exon 2 of the wild type surfactant protein C gene.

30. The method of claim 24, wherein the surfactant protein C null allele is created by a deletion of at least 50 consecutive nucleotides of coding sequence of the surfactant protein C gene.

31. The transgenic mammal of claim 4, wherein the mammal is an SP-C knockout mouse.

32. The transgenic mammal of claim 31, wherein the mammal is a proSP-C knockout.

33. The transgenic mammal of claim 4, wherein the mammal does not express SP-C.

34. The transgenic mammal of claim 4, wherein the mammal does not express active SP-C.

35. The method of claim 24 wherein the mouse is an SP-C knockout mouse.

36. The method of claim 35 wherein the mouse is a proSP-C knockout.

37. The method of claim 24 wherein the mouse does not express SP-C.

38. The method of claim 24 wherein the mouse does not express active SP-C.

39. A method of treating pulmonary disease in a subject comprising the administration to a subject in need of such treatment a therapeutically effective amount of a formulation comprising a SP-C therapeutic.

40. The method of claim 1 wherein the SP-C therapeutic is an agent selected from the group consisting of an isolated SP-C protein, an isolated nucleic acid molecule encoding a SP-C protein, a SP-C receptor-specific antibody that stimulates the activity of the receptor, or pharmaceutically acceptable composition thereof.

41. The method of claim 40, wherein the SP-C therapeutic agent is a SP-C receptor-specific antibody that stimulates the activity of the receptor.

42. The method of claim 40, wherein the SP-C therapeutic agent is an isolated SP-C protein or proSP-C protein.

43. The method of claim 40, wherein the SP-C therapeutic agent is an isolated nucleic acid molecule encoding a SP-C protein or proSP-C protein, wherein the nucleic acid molecule is operatively linked to a transcription control sequence.

44. The method of claim 43, wherein the nucleic acid molecule is expressed in the subject's airway cells.

45. The method of claim 44, wherein the nucleic acid that encodes a SP-C polypeptide, fragment, homolog or variant with substantial homology, supplying SP-C function.

46. The method of claim 45, wherein the nucleic acid molecule becomes integrated to the chromosomal DNA making up the genome of the subject's airway cells.

47. The method of claim 45, wherein the nucleic acid molecule is expressed by the subject's airway cells from an extrachromosomal location.

48. The method of claim 45, wherein the nucleic acid molecule comprises at least 50 nucleotides.

49. The method of claim 45, wherein the nucleic acid molecule comprises at least 200 nucleotides.

50. The method of claim 45, wherein the airway cells are selected from the group consisting of smooth muscle and epithelial cells.

51. The method of claim 45, wherein the isolated nucleic acid molecule is administered to the mammal complexed with a liposome delivery vehicle.

52. The method of claim 45, wherein the isolated nucleic acid molecule is administered to the mammal in a viral vector delivery vehicle.

53. The method of claim 52, wherein the viral vector delivery vehicle is from adenovirus.

54. The method of claim 45, wherein the isolated nucleic acid molecule, when administered to the lungs of the mammal, is expressed in cells of the mammal.

55. The method of claim 40, wherein the disease is a chronic obstructive pulmonary disease of the airways associated with eosinophilic inflammation.

56. The method of claim 40, wherein the disease is selected from the group consisting of airway obstruction, allergies, asthma, acute inflammatory lung disease, chronic inflammatory lung disease, chronic obstructive pulmonary dysplasia, emphysema, pulmonary emphysema, chronic obstructive emphysema, adult respiratory distress syndrome, bronchitis, chronic bronchitis, chronic asthmatic bronchitis, chronic obstructive bronchitis, and interstitial lung diseases.

57. The method of claim 40, wherein the SP-C therapeutic agent decreases lung inflammation in the mammal.

58. The method of claim 40, wherein the SP-C therapeutic agent is administered in an amount between about 0.1 micrograms/kilogram and about 10 milligram/kilogram body weight of a mammal.

59. The method of claim 40, wherein the SP-C therapeutic agent is administered in a pharmaceutically acceptable excipient.

60. The method of claim 40, wherein the mammal is a human.

61. The method of claim 1, wherein the SP-C therapeutic agent is administered by at least one route selected from the group consisting of nasal and inhaled routes.

62. The method of claim 40, wherein the pulmonary disease is selected from the group consisting of asthma, allergic bronchopulmonary aspergillosis, hypersensitivity pneumonia, eosinphilic pneumonia, allergic bronchitis bronchiectasis, hypersensitivity pneumotitis, occupational asthma, reactive airway disease syndrome, hypereosinophilic syndrome, rhinitis, sinusitis, and parasitic lung disease.

63. A method for prescribing treatment for airway hyperresponsiveness and/or airflow limitation associated with a respiratory disease involving an inflammatory response in a mammal, comprising: a. administering to the lungs of a mammal a SP-C therapeutic agent selected from the group consisting of: a SP-C receptor-specific antibody that stimulates the activity of the receptor an isolated SP-C protein or proSP-C protein; and an isolated nucleic acid molecule encoding a SP-C protein or proSP-C protein, wherein the nucleic acid molecule is operatively linked to a transcription control sequence; b. measuring a change in lung function in response to a provoking agent in the mammal to determine if the SP-C therapeutic agent modulates airway hyperresponsiveness; and c. prescribing a pharmacological therapy comprising administration of SP-C therapeutic agent to the mammal effective to reduce inflammation based upon the changes in lung function.

64. A formulation for protecting a mammal from airway hyperresponsiveness, airflow limitation and/or airway fibrosis associated with a respiratory disease involving inflammation, comprising an anti-inflammatory agent effective for reducing eosinophilic inflammation and a SP-C therapeutic agent selected from the group consisting of: a SP-C receptor-specific antibody that stimulates the activity of the receptor; an isolated SP-C protein or proSP-C protein; and an isolated nucleic acid molecule encoding a SP-C protein or proSP-C protein, wherein the nucleic acid molecule is operatively linked to a transcription control sequence.

65. The formulation of claim 64, wherein the formulation comprises a pharmaceutically acceptable excipient.

66. The formulation of claim 64, wherein the formulation comprises a controlled release vehicle selected from the group consisting of biocompatible polymers, other polymeric matrices, capsules, microcapsules, microparticles, bolus preparations, osmotic pumps, diffusion devices, liposomes, lipospheres, viral vectors and transdermal delivery systems.

67. The formulation of claim 64, wherein the SP-C therapeutic agent is an isolated SP-C protein or proSP-C protein.

68. The formulation of claim 64, wherein the SP-C therapeutic agent is an isolated nucleic acid molecule encoding a SP-C protein or proSP-C protein, wherein the nucleic acid molecule is operatively linked to a transcription control sequence.

69. The formulation of claim 68, wherein the isolated nucleic acid molecule is complexed with a liposome delivery vehicle.

70. The formulation of claim 68, wherein the isolated nucleic acid molecule in a viral vector delivery vehicle.

71. The formulation of claim 70, wherein the viral vector delivery vehicle is from adenovirus.

72. The formulation of claim 64, wherein the SP-C therapeutic agent is a SP-C receptor-specific antibody that stimulates the activity of the receptor.

73. The formulation of claim 64, wherein the SP-C therapeutic agent is selected from the group consisting of: an isolated SP-C protein or proSP-C protein and an isolated nucleic acid molecule encoding a SP-C protein or proSP-C protein, wherein the nucleic acid molecule is operatively linked to a transcription control sequence.

74. The formulation of claim 64, wherein the anti-inflammatory agent is selected from the group consisting of anti-IgE, immunomodulating drugs, leukotriene synthesis inhibitors, leukotriene receptor antagonists, glucocorticosteroids, steroid chemical derivatives, anti-cyclooxygenase agents, beta-adrenergic agonists, methylxanthines, cromones, anti-CD4 reagents, anti-IL-5 reagents, surfactants, cytoxin, and heparin.

75. The formulation of claim 64, wherein the anti-inflammatory agent is selected from the group consisting of leukotriene synthesis inhibitors, leukotriene receptor antagonists, glucocorticosteroids, beta-adrenergic agonists, methylxanthines, and cromones.


SEQUENCE LISTING (6 sequences)

SEQ ID NO: 1 - DNA, 3633 bp, Mus musculus
  gene (321)..(3443)
  mRNA (321)..(3443): join (321..389, 1619..1777, 2112..2234, 2472..2582, 2911..3066, 3268..3443)
  exon (321)..(389)
  Intron (390)..(1618)
  exon (1619)..(1777)
  Intron (1778)..(2111)
  exon (2112)..(2234)
  Intron (2235)..(2471)
  exon (2472)..(2582)
  Intron (2583)..(2910)
  exon (2911)..(3066)
  Intron (3067)..(3267)
  exon (3268)..(3443)
  ref <308>: DATABASE ACCESSION NUMBER: M38314
  ref <309>: DATABASE ENTRY DATE: 1999-07-28
  ref <313>: RELEVANT RESIDUES: (1)..(3633)
ctgcaggggcaggtgccagcaagagaggcagacatgcagaaagacacccacggagagaggagcaggtggcccgagggtgagtttgcttgacctcacccaggtttgctcttgttggggccaagaggattcatgtgcctaggccaagggccttgggggctcttgcagctgccttatcggggcctgggctctgaaaagccaggaacaaacaagctacaaagccaaggacttggctggcagacaggaggcccagtccttcacccctgtcctctctgtctctgatgatatatataagacactggtcacaccagagagatgaggagaggagagagagagagaaaccttacaaaatggacatgagtagcaaagaggtcctgatggagagtccaccggtgagtgtgattgtgtgtgtgtgtgtgtgtgctgcgcgcgcatacatagtatactgacttgactgtccatcctccagtaggctttttttctttttctagacccatatactcaattagcttaggattgggcttttaggatagagataccgtgatcattaagagtccctggtagaggcaggcacgacgaccacgaattcctgtataaagggattgagagccacagaggggtcaatagaaagtcctgtgaagaacactgatagcaccaccatgtagttgggaggctacaggaagtaccacagagcaaatgacagtgaggctgggatgtagatcagtagctagagcactttgcaaaacgtggagaccctggggtttcagtcccccagatccacataatccaggcatagaggtgcacactgtttggaaatataagcaagataatcattcagggtcatccttagatatataaggagttgaaaggcagcctgggtcatagatatggtttttttctttaaaaaaaaaaaaaggatggctcagtaggtagtggtgcttgcaccaaatgtggcggctcgagtttaatctctggaattcacatgatgaaaggagatgaaaagttgtccctgacttacacacacacatgtacatacacactcacacatactacacacatgtacacaaacattcacactcacaccacatacacacactctcacacacaccacacacatgtgcacacacacacacacacatgtaccttcacacacatcacacacatgcacactcaaacataccacacaatgtatacacgctcacacactaaatccgtgtaaaaaaccatgaagctggcacatggactggattgagaaggaacaaaggagacccgtgtaaaggtatagagacagggctacctggatcctttgagggagcaaaaactcaggatatgtctggggtgttaggcgtcgtgggaaaacagaagaagagatcggtagctggctctcttggattatttcaaacctagactttgcttctaactttctaagtagctatggttttgttagggtccaaccaccttccccaaagccttccttcccaaaagcctctgatctccccagctcttccttcacactcgtagcctaaagaggtacgggatgtgtgtgtcccccttgcacagggagagtaggagccaccctctccactacccacttgtctctctgcttcccttgctctctcaggattactcggcaggtcccaggagccagttccgcatcccctgctgtcccgtgcacctcaaacgccttctcatcgtggttgtggtggtggtcctcgttgtcgtggtgattgtaggggctctgctcatgggcctccacatgagtcaaaaacatactgagatggtgagtgggcctgggttgggcaaagaggcacagcagacagggggttgggggagattatgggggatgggcagctgttcggaggaagagaagggagtggacaggtatgagcacatcttgggtgacacaaacagagacgaggtagccatcctgcctagatcctctcccccagccccggcctagtgtgataaccatcgattgctctgacacctcttacgtttagccttcctgagatctcaggaaagcgtttgaatagaggattctgagtagatatgggtaccgaaagctgagggaagaaagagaaataccaggcagcattccaacacccttcccctaggtccttgagatgagcatcggagcaccggaaactcagaaacgcctagccccgagtgagcgagcagacaccatcgctaccttttccatcggctccactggcatcgttgtgtatgactaccagcgggtgaggatgccggaggaccaccgggactttattggaactagccagttgtagcatttctagaggtctctccccattctgtgcctggctacctcacctcagatgctcgaaccactgacgcaagtgcgcccctccaccctctgaagacaatctaaaggaagttggttggctgagaactagggttggggaggaagcaaggcaaggggaccttgtgaatgacctccagggttttatacctagctcctgacggcctataagccagctccaggaacctactgctacatcatgaagatggctccagagagcatccctagtcttgaggctttcgctagaaaactccagaacttcaggtgggtatgtttagggagggagggagcagtctcctctgaggtttgagtagaagggacatgtgaaagatgactagcgtaccctgtgtagtattgatgtttctgatcagacatgttctcctctctccatgacctgtgtcctgccatccctaccagctctcaggtggccctgctaagttgttggccttggctgagcttagacatgacctatgggcttctacatccaacccagtccctctctgaatttgtgaggaaactgatcctcgagaattaccaacttagtgtcccacactaaataaagcaggtgacattgaaagtaggtgttctttccagccaagccctccacacccacctctaagctgggccaggaggaagggcatgatactggttccgagtccgattcttccgggagagacctggctttcctaggccttgctgtgagcaccctgtgtggagagctaccactctactatatctagcatccacaggtgagcaacagtacctttcagggtgcctgggcaacactggcagggcttgggctgcctgctttgtcaggggacctactaggtatctcttaaagtcagtggtctcgggagctcggaggatggagggttcccagacacatcccacactggacccaagcgggtggcttcttcagtccccataagcattagttctttgcttcacagggtcggtagaaaccgcagcgggacaggaaagaccctccgcaaagggtctttgtcagacaagcaggaagctgctcctgcccagaaaccggtggaagtctgtaaaggaaaggtgtctctcctacgggccagggggatcctatcacaaaagaataaagcagcctgattggaaaacaaagagtggcgcttcttttctttcacattttctcagctcggcttctagcagaagctcctaggaaggagagggtttggagagttgggcgctttgcacgtcctttctaagagtgatggaggtttggtctaccccaggtaatgggaacagagcaaaaggcagggacagaggacgggacctgggtgccatacacgggg

SEQ ID NO: 2 - DNA, 995 bp, Mus musculus
aggagagagagagagaaaccttacaaaatggacatgagtagcaaagaggtcctgatggagagtccaccggattactcggcaggtcccaggagccagttccgcatcccctgctgtcccgtgcacctcaaacgccttctcatcgtggttgtggtggtggtcctcgttgtcgtggtgattgtaggggctctgctcatgggcctccacatgagtcaaaaacatactgagatggtccttgagatgagcatcggagcaccggaaactcagaaacgcctagccccgagtgagcgagcagacaccatcgctaccttttccatcggctccactggcatcgttgtgtatgactaccagcggctcctgacggcctataagccagctccaggaacctactgctacatcatgaagatggctccagagagcatccctagtcttgaggctttcgctagaaaactccagaacttcagggccaagccctccacacccacctctaagctgggccaggaggaagggcatgatactggttccgagtccgattcttccgggagagacctggctttcctaggccttgctgtgagcaccctgtgtggagagctaccactctactatatctagcatccacaggtgagcaacagtacctttcagggtgcctgggcaacactggcagggcttgggctgcctgctttgtcaggggacctactaggtatctcttaaagtcagtggtctcgggagctcggaggatggagggttcccagacacatcccacactggacccaagcgggtggcttcttcagtccccataagcattagttctttgcttcacagggtcggtagaaaccgcagcgggacaggaaagaccctccgcaaagggtctttgtcagacaagcaggaagctgctcctgcccagaaaccggtggaagtctgtaaaggaaaggtgtctctcctacgggccagggggatcctatcacaaaagaataaagcagcctgattggaaaacaaag

SEQ ID NO: 3 - protein, 193 aa, Mus musculus
  ref <308>: DATABASE ACCESSION NUMBER: AAA40010
  ref <309>: DATABASE ENTRY DATE: 1999-07-28
  ref <313>: RELEVANT RESIDUES: (1)..(193)
MDMSSKEVLMESPPDYSAGPRSQFRIPCCPVHLKRLLIVVVVVVLVVVVIVGALLMGLHMSQKHTEMVLEMSIGAPETQKRLAPSERADTIATFSIGSTGIVVYDYQRLLTAYKPAPGTYCYIMKMAPESIPSLEAFARKLQNFRAKPSTPTSKLGQEEGHDTGSESDSSGRDLAFLGLAVSTLCGELPLYYI

SEQ ID NO: 4 - DNA, 3409 bp, Homo sapiens
  gene (591)..(3237)
  mRNA (591)..(3237): join(616..657,1356..1514,1860..1982,2206..2316, 2651..2809); gene=“SP-C1”; product=“pulmonary surfactant protein SP-C”
  mRNA (591)..(3237): join(616..657,1356..1514,1860..1982,2206..2316, 2669..2809); gene=“SP-C1”; product=“pulmonary surfactant protein SP-C1”
  exon (591)..(657)
  Intron (658)..(1355)
  exon (1356)..(1514)
  Intron (1515)..(1859)
  exon (1860)..(1982)
  Intron (1983)..(2205)
  exon (2206)..(2316)
  Intron (2317)..(2650)
  exon (2651)..(3237)
  ref <308>: DATABASE ACCESSION NUMBER: J03890
  ref <309>: DATABASE ENTRY DATE: 1998-08-17
  ref <313>: RELEVANT RESIDUES: (1)..(3409)
ggtaccagatatgtgggaggaggcaaggtaagggaaagagtacttgaagttggaactggtccttgcagggaaatgcacatttatgaaaccccgaaaactgatgtcaaagcacctcctgccttgggcagtcctctcagagtctacaggtgctgcctccagaaccctcttcctggagcgcatccctatgtatctagaaattctgctgggaaatatgatggtcagacccttggccacctgaaagttcagggtggtagaagaaaaaggaaagccacagggcagcaggggcaggtgcagcaaggaaggcaggcacgccaggaagacacccatgggtagaagtgcagatggcccgagggcacagtttgctcaactcacccaggtttgctcttgctggggccaagaggactcatgtgccagggccaagggctctgggggctctcacagggggcttatctgggcttcggttctggagggccaggaacaaacaggcttcaaagcaagggcttggctggcacacaggggcttggtccttcacctctgtccctctcctacggacacatataagaccctggtcacacctgggagaggaggagaggagagcatagcacctgcagcaagatggatgtgggcagcaaagaggtcctgatggagagcccgccggtgagtgtggttgcgtgtgtgtatgtatgtgcgcgcgcacatgtgtgtgatggccctgcctcctctatcctccctggcctgtttccttatccagatccattcactcaactaacctaggactgtgataagtcaggatggggacaccaagaccactaagccagggacccttggggagctgtttgtggccaagagccactataggggtccgtagaactggagtgcgcgtagacagccctgagtcagaagccatgagaaacttcagaagtcaggggacacttctcagagaaaaaccacatacgagctggagccagaataaggaggagctcgcccggtggagaaggaggaaggcattccaggaaggagggagactctgtatcaccgcatggaggtgatcacttggggagagagaggggctgaccatggctgggggaagcagcagggagagacaggtgaagcaggctctcttgggtccctcaaaactagaccctgcttctaagcttctatgtatctatgggtttgttagaatccaggccacctcctccaagaagccttctctgatctcctcagcccttccctgtccatccatcgcatcggctgtccagcctaggagccgtgggagggtgttcagcttgtatagggagaagaggggacagcctcatgacctcatgcctgtctccttgcctgccccaccgtgtcaggactactccgcagctccccggggccgatttggcattccctgctgcccagtgcacctgaaacgccttcttatcgtggtggtggtggtggtcctcatcgtcgtggtgattgtgggagccctgctcatgggtctccacatgagccagaaacacacggagatggtgagaggtgtgggatgcacagcagtgggcacaggacatgccagacagaggggctaggtgggatgggcgataggaaactgtccaaggggagtggaggggaggaggcaaggggcacagctagaaggaaagaggcacgaaccaggcagcaacccagctcaggcttttccacaaggcccctgcccgcgacaggacagccagctccctccagcacctggttccactcagcctccctgaactcttgggaaagagggaagcgcatttgagtacagaggcctgagtatggggatgggtaccactggctgagtaggaaaggggaagaccaggtggctccatgcctttccccaggttctggagatgagcattggggcgccggaagcccagcaacgcctggccctgagtgagcacctggttaccactgccaccttctccatcggctccactggcctcgtggtgtatgactaccagcaggtgggtatgccagacctcctgacctggaccaatgacaactgggctctgctagagcgcccagctggccactttcattccacatccatctctcctctctcagactttttgctgagcccagattctagtagtctcccgtgcccaacctagagggaggtggctaaggacctgggtcagggagagagcagggcaggaccccgaatgatctccagcattctgtgcctagctgctgatcgcctacaagccagcccctggcacctgctgctacatcatgaagatagctccagagagcatccccagtcttgaggctctcactagaaaagtccacaacttccaggtgtgtgtgtgtgggtgaaaagagtgggctgtctccctcccaggctgctggaggagtgtccgaatggtggctatttgtcacctgtaaagcactgttcctcattggctgccagctgactgcccctctcctattcccctgcacgactcctttccttcccaccccactgccaagctgctgggctcagctgagtccactcactacctggtggcttctgactctagcacagcccctctttactgatgagaaaactgaggctcagagagattgcctgatatacctgaagtcccacaataagggctgcacatgggatagaaactcacttcctacattccagatggaatgctctctgcaggccaagcccgcagtgcctacgtctaagctgggccaggcagaggggcgagatgcaggctcagcaccctccggaggggacccggccttcctgggcatggccgtgaacaccctgtgtggcgaggtgccgctctactacatctaggacgcctccggtgagcaggtgtgatcccagggcccctgatcagcagcggaggagcgctggccacctgcccggctgtggaggaggctcgctgaccaggctggggcgtccactgaagcggggtcatccaggcaactcgggggaggggaagctcacagaccggtacttcccactcccctgaattctctctgtccatcctcaacattcctttgcttcatagggtcagtggaagccccaacggaaaggaaacgccccgggcaaagggtcttttgcagcttttgcagacgggcaagaagctgcttctgcccacaccgcagggacaaaccctggagaaatgggagcttggggagaggatgggagtgggcagaggtggcacccaggggcccgggaactcctgccacaacagaataaagcagcctgatttgaaaagcaaagggtctgcttctgtcttcctgcagggcgcagtcctcgctggcggggccggccaagaagggaagggccttgggagagcaaagtggggtttccattcgccctctgtcccagggcgctggcactgtccacctcggcggggagaggggctcgcagggagcatccacgggcttt

SEQ ID NO: 5 - protein, 197 aa, Homo sapiens [sequence omitted]

SEQ ID NO: 6 - protein, 191 aa, Homo sapiens [sequence omitted]